(19) United States
(12) Patent Application Publication
(10) Pub. No.: US 2019/0178900 A1
(43) Pub. Date: Jun. 13, 2019

(54) AUTISM SUBSETS

(71) Applicant: Stemina Biomarker Discovery, Inc.

(72) Inventors: Alan Smith, Madison, WI (US); Paul West, Arena, WI (US); Robert Burrier, Verona, WI (US); Joseph King, Madison, WI (US); Roxana Alexandridis, Madison, WI (US); Laura Egnash, Deforest, WI (US); Elizabeth L.R. Donley, Madison, WI (US); Michael Ludwig, Middleton, WI (US)

(73) Assignees: Stemina Biomarker Discovery, Inc., Madison, WI (US); Stemina Biomarker Discovery, Inc., Madison, WI (US)

(21) Appl. No.: 16/301,044

(22) PCT Filed: May 12, 2017
(86) PCT No.: PCT/US17/32386

§ 371 Date: Nov. 13, 2018

Related U.S. Application Data
(60) Provisional application No. 62/336,134, filed on May 13, 2016.

(51) Int. Cl. G01N 33/68; G01N 30/72

(52) U.S. Cl. CPC G01N 33/6896; G01N 30/72; G01N 2570/00; G16B 40/00; G01N 2800/52; G01N 2800/60; G01N 2800/28

(57) ABSTRACT

With the present invention, metabolomics biomarkers are used to identify subtypes within the autism spectrum disorder (ASD) population. In one embodiment, levels of the metabolite 3-carboxy-4-methyl-5-propyl-2-furanpropanoic acid (CMPF) of about six times or greater than the median level in typically developing (TD) individuals or about 2 μM or greater place the individual in an autism subpopulation that includes less than 20% of the total ASD population. Thus, CMPF is a biomarker able to discriminate a subtype of ASD.

### CONTINUING APPLICATION DATA

This application claims the benefit of U.S. Provisional Application Ser. No. 62/336,134, filed May 13, 2016, which is incorporated by reference herein.

### GOVERNMENT FUNDING

This invention was made with government support under Grant No. 1R44MH107124-01, awarded by the National Institute of Mental Health, National Institutes of Health. The Government has certain rights in the invention.

### BACKGROUND

Autism is a complex spectrum of neurodevelopmental disorders with heterogeneous underlying genetic, metabolic, and environmental causes that may lead to individual differences in response to therapies. A recent study, based on 2014 data, estimated the prevalence of autism spectrum disorder (ASD) in children ages 3 to 17 in the United States at 2.24% (1 in 45 children) (Zablotsky et al., 2015, “Estimated prevalence of autism and other developmental disabilities following questionnaire changes in the 2014 National Health Interview Survey,” National Health Statistics Reports; No 87. Hyattsville, Md.: National Center for Health Statistics). The diagnosis of ASD at the earliest age possible is important for effective intervention. Earlier diagnosis of children with ASD improves outcomes by providing therapeutic interventions that lead to higher cognitive and social function as well as improved communication, subsequently decreasing the financial and emotional burden on families and society (Dawson et al., 2010, Pediatrics; 125: e17-23; and Ganz, 2007, Arch Pediatr Adolesc Med; 161: 343-349). There is a need for reliable biomarker-based tests that support the etiological diagnosis of autism that are amenable to both the idiopathic and syndromic forms of autism and can aid selection of an efficacious therapy.

### SUMMARY OF THE INVENTION

The present invention includes a method of diagnosing autism, the method including measuring the level of 3-carboxy-4-methyl-5-propyl-2-furanpropanoic acid (CMPF) in a biosample obtained from the individual; wherein a level of CMPF at a level that is at least about six times or more than the median level in typically developing (TD) individuals indicates autism, and/or wherein a level of CMPF of at least about 2 μM or greater indicates autism.

In some aspects, the method further includes measuring the level of one or more metabolites selected from 3-hydroxy-3-methylbutyric acid, 3-methyl-2-oxovaleric acid, salicylic acid, gentisic acid, a CMPF-related metabolite, DHEA sulfate, pregnenolone sulfate, LysoPE(22:6), glycine, 1-alanine, sacrosine, and/or proline betaine.

In some aspects, 3-hydroxy-3-methylbutyric acid of a fold change range of ASD/TD of about 0 to about 0.84, 3-methyl-2-oxovaleric acid of a fold change range of ASD/TD of about 0 to about 0.87, salicylic acid of a fold change range of ASD/TD of about 0 to about 0.77, gentisic acid of a fold change range of ASD/TD of about 0 to about 0.71, and/or proline betaine of a fold change range of ASD/TD of about 0 to about 0.72 is indicative of autism.

In some aspects, the CMPF-related metabolite of a fold change range of TD/ASD of about 0 to about 0.12, DHEA sulfate of a fold change range of TD/ASD of about 0 to about 0.38, pregnenolone sulfate of a fold change range of TD/ASD of about 0 to about 0.58, LysoPE(22:6) of a fold change range of TD/ASD of about 0 to about 0.78, glycine of a fold change range of TD/ASD of about 0 to about 0.75, 1-alanine of a fold change range of TD/ASD of about 0 to about 0.86, sacrosine of a fold change range of TD/ASD of about 0 to about 0.85 is indicative of autism.

In some aspects, 3-hydroxy-3-methylbutyric acid of a fold change range of ASD/TD is less than 0.84, 3-methyl-2-oxovaleric acid of a fold change range of ASD/TD of less than 0.87, salicylic acid of a fold change range of ASD/TD of less than 0.77, gentisic acid of a fold change range of ASD/TD of less than 0.71, the CMPF-related metabolite of a fold change range of ASD/TD of greater than about 8.43, DHEA sulfate of a fold change range of ASD/TD of greater than about 2.63, pregnenolone sulfate of a fold change range of ASD/TD of greater than about 1.71, LysoPE(22:6) of a fold change range of ASD/TD of greater than about 1.38, glycine of a fold change range of ASD/TD of greater than about 1.34, 1-alanine of a fold change range of ASD/TD of greater than about 1.17, sacrosine of a fold change range of ASD/TD of greater than about 1.17, and/or proline betaine of a fold change range of ASD/TD of greater than about 0.72 is indicative of autism.

In some aspects, the measurement of 3-hydroxy-3-methylbutyric acid is less than 0.84 times the TD average or 1/0.84 times greater than the TD average, the measurement of 3-methyl-2-oxovaleric acid is less than 0.87 times the TD average or 1/0.87 times greater than the TD average, salicylic acid is less than 0.77 the TD average or 1/0.77 greater than the TD average, gentisic acid is less than 0.71 times the TD average or 1/0.71 times greater than the TD average, the CMPF-related metabolite is less than 8.43 times the TD average or 1/8.43 times greater than the TD average, DHEA sulfate is less than 2.63 times the TD average or 1/2.63 times greater than the TD average, pregnenolone sulfate is less than 1.71 times the TD average or 1/1.71 times greater than the TD average, LysoPE(22:6) is less than 1.38 times the TD average or 1/1.38 times greater than the TD average, glycine is less than 1.34 times the TD average or 1/1.34 times greater than the TD average, 1-alanine is less than 1.17 times the TD average or 1/1.17 times greater than the TD average, sacrosine is less than 1.17 times the TD average or 1/1.17 times greater than the TD average, and/or proline betaine is less than 0.72 times the TD average or 1/0.72 times greater than the TD average is indicative of autism.

The present invention includes a method of diagnosing autism, the method including measuring the level of one or more metabolites selected from 3-hydroxy-3-methylbutyric acid, 3-methyl-2-oxovaleric acid, salicylic acid, gentisic acid, a CMPF-related metabolite, DHEA sulfate, pregnenolone sulfate, LysoPE(22:6), glycine, 1-alanine, sacrosine, and/or proline betaine in a biosample obtained from the individual; wherein 3-hydroxy-3-methylbutyric acid of a fold change range of ASD/TD of about 0 to about 0.84, 3-methyl-2-oxovaleric acid of a fold change range of ASD/TD of about 0 to about 0.87, salicylic acid of a fold change range of ASD/TD of about 0 to about 0.77, gentisic acid of a fold change range of ASD/TD of about 0 to about 0.71, and/or proline betaine of a fold change range of ASD/TD of about 0 to about 0.72 is indicative of autism; and/or the CMPF-related metabolite of a fold change range of TD/ASD of about 0 to about 0.12, DHEA sulfate of a fold change range of TD/ASD of about 0 to about 0.38, pregnenolone sulfate of a fold change range of TD/ASD of about 0 to about 0.58, LysoPE(22:6) of a fold change range of TD/ASD of about 0 to about 0.78, glycine of a fold change range of TD/ASD of about 0 to about 0.75, 1-alanine of a fold change range of TD/ASD of about 0 to about 0.86, sacrosine of a fold change range of TD/ASD of about 0 to about 0.85 is indicative of autism; and/or 3-hydroxy-3-methylbutyric acid of a fold change range of ASD/TD is less than 0.84, 3-methyl-2-oxovaleric acid of a fold change range of ASD/TD of less than 0.87, salicylic acid of a fold change range of ASD/TD of less than 0.77, gentisic acid of a fold change range of ASD/TD of less than 0.71, the CMPF-related metabolite of a fold change range of ASD/TD of greater than about 8.43, DHEA sulfate of a fold change range of ASD/TD of greater than about 2.63, pregnenolone sulfate of a fold change range of ASD/TD of greater than about 1.71, LysoPE(22:6) of a fold change range of ASD/TD of greater than about 1.38, glycine of a fold change range of ASD/TD of greater than about 1.34, 1-alanine of a fold change range of ASD/TD of greater than about 1.17, sacrosine of a fold change range of ASD/TD of greater than about 1.17, and/or proline betaine of a fold change range of ASD/TD of greater than about 0.72 is indicative of autism; and/or a measurement of 3-hydroxy-3-methylbutyric acid is less than 0.84 times the TD average or 1/0.84 times greater than the TD average, the measurement of 3-methyl-2-oxovaleric acid is less than 0.87 times the TD average or 1/0.87 times greater than the TD average, salicylic acid is less than 0.77 the TD average or 1/0.77 greater than the TD average, gentisic acid is less than 0.71 times the TD average or 1/0.71 times greater than the TD average, the CMPF-related metabolite is less than 8.43 times the TD average or 1/8.43 times greater than the TD average, DHEA sulfate is less than 2.63 times the TD average or 1/2.63 times greater than the TD average, pregnenolone sulfate is less than 1.71 times the TD average or 1/1.71 times greater than the TD average, LysoPE(22:6) is less than 1.38 times the TD average or 1/1.38 times greater than the TD average, glycine is less than 1.34 times the TD average or 1/1.34 times greater than the TD average, 1-alanine is less than 1.17 times the TD average or 1/1.17 times greater than the TD average, sacrosine is less than 1.17 times the TD average or 1/1.17 times greater than the TD average, and/or proline betaine is less than 0.72 times the TD average or 1/0.72 times greater than the TD average is indicative of autism

The present invention includes a method of placing an individual within an autism subpopulation, the method including measuring the level of 3-carboxy-4-methyl-5-propyl-2-furanpropanoic acid (CMPF) in a biosample obtained from the individual; wherein a level of CMPF at a level that is at least about six times or more than the median level in TD individuals and/or a level of CMPF of at least about 2 μM or greater places the individual in a CMPF associated autism subpopulation.

The present invention includes a method of placing an individual previously clinically diagnosed with autism spectrum disorder (ASD) in an autism subset, the method including obtaining a biosample from the individual; quantifying the concentration amount of 3-carboxy-4-methyl-5-propyl-2-furanpropanoic acid (CMPF) in the biosample; wherein if the concentration of CMPF is about 2 μM or greater and/or at least about six times or greater than the median level in TD individuals, then placing the individual in a CMPF associated autism subpopulation; and/or if the concentration of CMPF is less than about 2 μM and/or less than about six times the median level in TD individuals, then the individual is not placed in a CMPF associated autism subset.

The present invention includes a method of diagnosing and treating an individual, the method including obtaining a biosample from the individual; quantifying the concentration amount of 3-carboxy-4-methyl-5-propyl-2-furanpropanoic acid (CMPF) in the biosample; wherein if the concentration of CMPF in the biosample is about 2 μM or greater and/or at least about six times or more than the median level in TD individuals, then administering an appropriate treatment; and/or if the concentration of CMPF in the biosample is less than about 2 μM and/or less than about six times the median level in TD individuals, then not administering an autism treatment.

The present invention includes a method of treating autism, the method including quantifying the concentration amount of 3-carboxy-4-methyl-5-propyl-2-furanpropanoic acid (CMPF) in a biosample obtained from an individual; wherein CMPF concentration is quantified using C18 (reverse phase) LC coupled with a triple quadrupole (QqQ) MS using electrospray ionization in the positive ion mode with analyte detection in the multiple reaction monitoring (MRM) mode and including a stable label internal standard and CMPF concentrations are measured distributed over a linear range of 0.05 to 100 μM; wherein if the concentration of CMPF is about 2 μM or greater and/or at least about six times or more than the median level in TD individuals, then administering an appropriate autism subset treatment; and/or if the concentration of CMPF is less than about 2 μM and/or less than about six times the median level in TD individuals, then not administering an appropriate autism subset treatment.

The present invention includes a method including obtaining a biosample from a human subject and quantifying the metabolite 3-carboxy-4-methyl-5-propyl-2-furanpropanoic acid (CMPF) in the biosample. In some aspects, the biosample is obtained from an individual with a neuro-developmental disorder. In some aspects, the biosample is obtained from developmentally delayed (DD) individual. In some aspects, the biosample is obtained from an individual with ASD. In some aspects, the concentration of CMPF in the biosample is about 2 μM or greater and/or at least about six times or more than the median level in TD individuals.

The present invention includes a method including obtaining a biosample from a human subject and measuring the metabolite 3-carboxy-4-methyl-5-propyl-2-furanpropanoic acid (CMPF) in the biosample; wherein CMPF concentrations are determined using C18 (reverse phase) LC coupled with a triple quadrupole (QqQ) MS using electrospray ionization in the positive ion mode with analyte detection in the multiple reaction monitoring (MRM) mode and including a stable label internal standard, wherein CMPF concentrations are measured distributed over a linear range of 0.05 to 100 μM.

The present invention includes a method including measuring by mass spectrometry the levels of a plurality of metabolites in a biosample obtained from a human subject, wherein the plurality of metabolites includes 3-carboxy-4-methyl-5-propyl-2-furanpropanoic acid (CMPF) and at least one metabolite selected from 3-hydroxy-3-methylbutyric acid, 3-methyl-2-oxovaleric acid, salicylic acid, gentisic acid, a CMPF-related metabolite, DHEA sulfate, pregnenolone sulfate, LysoPE(22:6), glycine, 1-alanine, sacrosine, proline betaine, 3-indoxyl sulfate, p-cresol sulfate, and/or a 3-omega fatty acid metabolite, such as for example, docosahexaenoic acid (DHA) or eicosapentaenoic acid (EPA).

The present invention includes a method of identifying a subpopulation within a population of individuals with autism spectrum disorder (ASD) individuals, the method including measuring the level of 3-carboxy-4-methyl-5-propyl-2-furanpropanoic acid (CMPF) in biosamples obtained from the population of individuals; and measuring the level of CMPF in biosamples obtained from a population of typically developing (TD) individuals; comparing the level of CMPF in biosamples obtained from ASD and/or DD individuals to the level of CMPF in biosamples obtained from TD individuals; wherein a level of CMPF at a level that is at least about six times or greater than the median level in TD individuals and/or a level of CMPF at least about 2 μM or greater places the ASD individuals in an autism subpopulation.

With any of the methods described herein, the method may further include measuring the level of one or more metabolites selected from 3-hydroxy-3-methylbutyric acid, 3-methyl-2-oxovaleric acid, salicylic acid, gentisic acid, a CMPF-related metabolite, DHEA sulfate, pregnenolone sulfate, LysoPE(22:6), glycine, 1-alanine, sacrosine, and/or proline betaine.

With any of the methods described herein, levels of CMPF and other metabolites in the biosample may be measured by mass spectrometry. In some aspects, mass spectrometry includes gas chromatography mass spectrometry (GC-MS), and liquid chromatography mass spectrometry (e.g. LC-MS, LC-MS-MS, LC-MRM, LC-SIM, LC-SRM) using reverse phase liquid chromatography coupled to electrospray ionization in positive ion polarity (C8pos), reverse phase liquid chromatography coupled to electrospray ionization in negative ion polarity (C8neg), hydrophilic interaction liquid chromatography coupled to electrospray ionization in positive ion polarity (HILICpos), and/or hydrophilic interaction liquid chromatography coupled to electrospray ionization in negative ion polarity (HILICneg).

In some aspects, mass spectrometry includes C18 (reverse phase) LC coupled with a triple quadrupole (QqQ) MS using electrospray ionization in the positive ion mode with analyte detection in the multiple reaction monitoring (MRM) mode and including a stable label internal standard, wherein CMPF concentrations are measured distributed over a linear range of 0.05 to 100 μM.

In some aspects, the determination of a metabolite may be by a methodology other than a physical separation method, such as for example, a colorimetric, enzymatic, immunological methodology, and gene expression analysis, including, for example, real-time PCR, RT-PCR, Northern analysis, and in situ hybridization.

With any of the methods described herein, the method may further include measuring the level of one or more additional non-CMPF uremic toxins; wherein the level of the one or more additional non-CMPF uremic toxins in the biosample obtained from the individuals in the autism subpopulation is similar to that in biosamples obtained from TD individuals. In some aspects, the one or more additional non-CMPF uremic toxin includes 3-indoxyl sulfate and/or p-cresol sulfate.

With any of the methods described herein, the method may further include measuring the level of one or more 3-omega fatty acid metabolites.

With any of the methods described herein, a biosample may be from an individual that does not suffer from uremia, type 2 diabetes, and/or gestational diabetes.

With any of the methods described herein, the method may further include measuring the level of one or more metabolites selected from 3-hydroxy-3-methylbutyric acid, 3-methyl-2-oxovaleric acid, salicylic acid, gentisic acid, a CMPF-related metabolite, DHEA sulfate, pregnenolone sulfate, LysoPE(22:6), glycine, 1-alanine, sacrosine, and/or proline betaine.

In some aspects, 3-hydroxy-3-methylbutyric acid of a fold change range of ASD/TD of about 0 to about 0.84, 3-methyl-2-oxovaleric acid of a fold change range of ASD/TD of about 0 to about 0.87, salicylic acid of a fold change range of ASD/TD of about 0 to about 0.77, gentisic acid fold change range of a ASD/TD of about 0 to about 0.71, and/or proline betaine of a fold change range of ASD/TD of about 0 to about 0.72 places the individual in the autism subpopulation. In some aspects, the CMPF-related metabolite of a fold change range of TD/ASD of about 0 to about 0.12, DHEA sulfate of a fold change range of TD/ASD of about 0 to about 0.38, pregnenolone sulfate of a fold change range of TD/ASD of about 0 to about 0.58, LysoPE(22:6) of a fold change range of TD/ASD of about 0 to about 0.78, glycine of a fold change range of TD/ASD of about 0 to about 0.75, 1-alanine of a fold change range of TD/ASD of about 0 to about 0.86, sacrosine of a fold change range of TD/ASD of about 0 to about 0.85 places the individual in the autism subpopulation. In some aspects, 3-hydroxy-3-methylbutyric acid of a fold ASD/TD is less than 0.84, 3-methyl-2-oxovaleric acid of a fold ASD/TD of less than 0.87, salicylic acid of a fold ASD/TD of less than 0.77, gentisic acid of a ASD/TD of less than 0.71, the CMPF-related metabolite of a fold ASD/TD of greater than about 8.43, DHEA sulfate of a fold ASD/TD of greater than about 2.63, pregnenolone sulfate of a fold ASD/TD of greater than about 1.71, LysoPE(22:6) of a fold ASD/TD of greater than about 1.38, glycine of a fold ASD/TD of greater than about 1.34, 1-alanine of a fold ASD/TD of greater than about 1.17, sacrosine of a fold ASD/TD of greater than about 1.17, and/or proline betaine of a fold ASD/TD of greater than about 0.72 places the individual in the autism subpopulation.

With any of the methods described herein, a biosample may be obtained from an individual previously diagnosed with autism spectrum disorder (ASD) and/or is undergoing treatment.

With any of the methods described herein, a biosample may be obtained from an individual not previously diagnosed with autism spectrum disorder (ASD).

With any of the methods described herein, a biosample includes cerebrospinal fluid, brain tissue, amniotic fluid, blood, serum, plasma, amniotic fluid, urine, breath condensate, sweat, saliva, tears, hair, cell membranes, and/or vitreous humour. In some aspects, a biosample includes plasma.

With any of the methods described herein, the subject may be an adult, a teenager, less than 13 years of age, less than 10 years of age, less than about 6 years of age, less than about 5 years of age, less than about 4 years of age, less than about 3 years of age, less than about 2 years of age, less than about 18 months of age, less than about 1 year of age, about 1 to about 6 years of age, about 1 to about 5 years of age, about 1 to about 4 years of age, about 1 to about 2 years of age, about 2 to about 6 years of age, about 2 to about 4 years of age, or about 4 to about 6 years of age.

With any of the methods described herein, the method may further include providing individualized treatment to the one or more individuals identified as belonging to a CMPF-associated autism subpopulation. In some aspects, individualized treatment includes modified diet, dietary supplements, probiotic therapy, and/or pharmacological therapy. In some aspects, individualized treatment includes administration of a CMPF inhibitor and/or angiotensin II AT1 receptor blocker. In some aspects, the method may further include quantifying the one or more metabolite indicative of ASD and/or an ASD subset at one or more time points after the initiation of treatment. In some aspects, the level of the one or more metabolites indicative of ASD and/or an ASD subset returns to TD levels after initiation of treatment.

The present invention includes a metabolomic signature for a subset of autism, the metabolomic signature including 3-carboxy-4-methyl-5-propyl-2-furanpropanoic acid (CMPF) at a concentration of at least about six times or greater than the median level in TD individuals and/or at least about 2 μM or greater. In some aspects, the metabolomic signature further includes at least one or metabolites selected from 3-hydroxy-3-methylbutyric acid, 3-methyl-2-oxovaleric acid, salicylic acid, gentisic acid, a CMPF-related metabolite, DHEA sulfate, pregnenolone sulfate, LysoPE(22:6), glycine, 1-alanine, sacrosine, and/or proline betaine. In some aspects, the metabolomic signature includes 3-hydroxy-3-methylbutyric acid of a fold change range of ASD/TD of about 0 to about 0.84, 3-methyl-2-oxovaleric acid of a fold change range of ASD/TD of about 0 to about 0.87, salicylic acid of a fold change range of ASD/TD of about 0 to about 0.77, gentisic acid of a fold change range of ASD/TD of about 0 to about 0.71, and/or proline betaine of a fold change range of ASD/TD of about 0 to about 0.72; and/or the CMPF-related metabolite of a fold change range of TD/ASD of about 0 to about 0.12, DHEA sulfate of a fold change range of TD/ASD of about 0 to about 0.38, pregnenolone sulfate of a fold change range of TD/ASD of about 0 to about 0.58, LysoPE(22:6) of a fold change range of TD/ASD of about 0 to about 0.78, glycine of a fold change range of TD/ASD of about 0 to about 0.75, 1-alanine of a fold change range of TD/ASD of about 0 to about 0.86, sacrosine of a fold change range of TD/ASD of about 0 to about 0.85; and/or 3-hydroxy-3-methylbutyric acid of a fold change range of ASD/TD is less than 0.84, 3-methyl-2-oxovaleric acid of a fold change range of ASD/TD of less than 0.87, salicylic acid of a fold ASD/TD of less than 0.77, gentisic acid of a fold change range of ASD/TD of less than 0.71, the CMPF-related metabolite of a fold change range of ASD/TD of greater than about 8.43, DHEA sulfate of a fold change range of ASD/TD of greater than about 2.63, pregnenolone sulfate of a fold change range of ASD/TD of greater than about 1.71, LysoPE(22:6) of a fold change range of ASD/TD of greater than about 1.38, glycine of a fold change range of ASD/TD of greater than about 1.34, 1-alanine of a fold change range of ASD/TD of greater than about 1.17, sacrosine of a fold change range of ASD/TD of greater than about 1.17, and/or proline betaine of a fold change range of ASD/TD of greater than about 0.72. In some aspects, the metabolic signature includes 3-hydroxy-3-methylbutyric acid is less than 0.84 times the TD average or 1/0.84 times greater than the TD average, the measurement of 3-methyl-2-oxovaleric acid is less than 0.87 times the TD average or 1/0.87 times greater than the TD average, salicylic acid is less than 0.77 the TD average or 1/0.77 greater than the TD average, gentisic acid is less than 0.71 times the TD average or 1/0.71 times greater than the TD average, the CMPF-related metabolite is less than 8.43 times the TD average or 1/8.43 times greater than the TD average, DHEA sulfate is less than 2.63 times the TD average or 1/2.63 times greater than the TD average, pregnenolone sulfate is less than 1.71 times the TD average or 1/1.71 times greater than the TD average, LysoPE(22:6) is less than 1.38 times the TD average or 1/1.38 times greater than the TD average, glycine is less than 1.34 times the TD average or 1/1.34 times greater than the TD average, 1-alanine is less than 1.17 times the TD average or 1/1.17 times greater than the TD average, sacrosine is less than 1.17 times the TD average or 1/1.17 times greater than the TD average, and/or proline betaine is less than 0.72 times the TD average or 1/0.72 times greater than the TD average.

In some aspects of the metabolic signature, CMPF concentrations are determined using C18 (reverse phase) LC coupled with a triple quadrupole (QqQ) MS using electrospray ionization in the positive ion mode with analyte detection in the multiple reaction monitoring (MRM) mode, and including a stable label internal standard wherein CMPF concentrations are measured distributed over a linear range of 0.05 to 100 μM.

The present invention includes a simple abundance threshold method of identifying one or more metabolites identifying a subpopulation within a population of individuals with autism spectrum disorder (ASD), or a method of identifying a subpopulation within a population of individuals with ASD, the method including:

measuring the levels of one or more features (for example, metabolites, putative-metabolites, unknown metabolites, proteins, and/or RNA) in two populations;

determining in one population an optimal upper threshold for each feature, wherein the upper threshold represents a level of the feature wherein all except about 1% of subjects with a TD diagnosis has a level of feature which is below the threshold;

counting the number of subjects with a diagnosis of ASD which have feature levels above the upper threshold and saving as a hypothetical diagnostic features where this count is above about 6% of the total number of ASD subjects;

repeating the above steps for all features wherein a lower threshold is used which represents a level for which all but one TD subject has levels above the lower threshold;

saving as a hypothetical diagnostic every feature for which about 6% or greater of ASD subjects have feature levels below the lower threshold;

creating a multitude of feature ratios for each hypothetical diagnostic by dividing the level determined for each subject by a set of normalizing features (this set can be all features or a selected set of features or determined metabolites or putative metabolites);

determining ratio optimal thresholds for each feature;

determining the percent of ASD subjects which have ratios above or below the thresholds, wherein ratios which distinguish the greatest number of ASD subjects are saved as diagnostic ratio;

using a second population of subjects wherein the age, demographics and collection conditions (for example fasted or non-fasted) can be the same or different from the first study;

and determining the performance of each diagnostic ratio using the same optimal threshold which was determined in the first population of subjects;

wherein ratio diagnostics which perform with greater than about 90% specificity and about 6% sensitivity (or any performance requirements one sees fit) reveal features which define a subtype in ASD.

In some aspects of the method of identifying one or more metabolites identifying a subpopulation within a population of individuals with autism spectrum disorder (ASD), or method of identifying a subpopulation within a population of individuals with ASD, one or more features include a confirmed metabolite. In some aspects, one or more features include a putative metabolite as determined by matching a library of features associated with known metabolites on mass and retention time. In some aspects, the first population and the second population include subsets of a single study. In some aspects, the first population and the second population includes two independent studies. In some aspects, feature levels and/or and diagnostic ratios are determined by the same or different techniques. In some aspects, feature levels and/or and diagnostic ratios are determined using mass spectrometry. In some aspects, wherein mass spectrometry includes gas chromatography mass spectrometry (GC-MS), and liquid chromatography mass spectrometry (e.g. LC-MS, LC-MS-MS, LC-MRM, LC-SIM, LC-SRM) using reverse phase liquid chromatography coupled to electrospray ionization in positive ion polarity (C8pos), reverse phase liquid chromatography coupled to electrospray ionization in negative ion polarity (C8neg), hydrophilic interaction liquid chromatography coupled to electrospray ionization in positive ion polarity (HILICpos), and/or hydrophilic interaction liquid chromatography coupled to electrospray ionization in negative ion polarity (HILICneg). In some aspects, mass spectrometry includes C18 (reverse phase) LC coupled with a triple quadrupole (QqQ) MS using electrospray ionization in the positive ion mode with analyte detection in the multiple reaction monitoring (MRM) mode including a stable label internal standard wherein CMPF concentrations are measured distributed over a linear range of 0.05 to 100 μM. In some aspects, selected features used in the denominator of the diagnostic ratio are selected to be non-complementary to the numerator or hypothetical diagnostic feature (i.e. is not itself a hypothetical diagnostic feature). In some aspects, the selected features used in the denominator are determined to be hypothetical diagnostics which when used in the denominator improve the diagnostic performance of the ratio (i.e. complementary to the feature in the numerator). In some aspects, one or more metabolite(s) used for the denominator is a spiked-in agent. In some aspects, the optimal upper threshold is defined as the level of feature for which all ASD or DD subjects are below the threshold and the optimal lower threshold is defined as the level of feature for all ASD or DD subjects which is above the threshold. In some aspects, the optimal lower and upper thresholds are: based on measures of dispersion population (for example, variance, IQR, MAD, CV, standard deviation, standard error and/or other statistical means) of the mean or median of the non-ASD; based on measures of dispersion population (for example, variance, IQR, MAD, CV, standard deviation, standard error and/or other statistical means) of the mean or median of the ASD population; based on the upper and lower quantiles of the non-ASD population; based on the upper and lower quantiles of the ASD population; based on a measure of statistical distance of the subjects with ASD to non-ASD subjects; based on a standard score or standardized variable of non-ASD subjects; based on a standard score or standardized variable of ASD subjects; and/or based on ROC AUC. In some aspects, the minimum percentage sensitivity required for the determination of a hypothetical diagnostic includes about 3%, about 4%, about 5%, about 7%, about 8%, about 9%, about 10%, about 11.%, about 12%, about 13%, about 14%, about 15%, about 16%, about 17%, about 18%, about 19%, or about 20%, rather than about 6%; wherein the ratio diagnostics perform with greater than at least about 95% specificity, at least about 96% specificity, at least about 97% specificity, at least about 98% specificity, or at least about 99% specificity; and/or wherein the ratio diagnostics perform with at least about 75% specificity, at least about 80% specificity, at least about 85% specificity, at least about 86% specificity, at least about 87% specificity, at least about 88% specificity, or at least about 89% specificity, rather than greater than about 90% specificity.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Box plot with scatter plot demonstrating the reproducibility of elevated CMPF levels among a subpopulation of ASD individuals (above the horizontal line) in two independent clinical studies. The y-axis corresponds to log base 2 transformed spiked-in internal standard normalized abundance values of the metabolite CMPF for the APP (left panel) and IMAGE (right panel) study subjects. Values of study subjects are represented as points. The upper horizontal line represents the diagnostic threshold that was determined in the APP study. The lower horizontal line represents the diagnostic threshold determined in the IMAGE study. The subjects with CMPF levels above this threshold exhibit the CMPF subtype. Points are colored red for ASD and black for typically developing.

FIG. 2. Scatterplot showing the relationship in abundance of the omega-3 fatty acid DHA and CMPF in the APP (left panel) and IMAGE (right panel) studies. Points correspond to subjects for ASD and TD. Subjects reporting omega-3 supplementation are solid and those not reporting omega-3 supplements are more transparent. The vertical line represents the diagnostic threshold value determined in the APP study implementing the 3 sigma rule. The angled lines correspond to the trend line fit using simple linear regression.

FIG. 3. Venn diagram combining autism subtypes for diagnostic panel.

FIG. 4. A diagram of metabolomic platform from sample preparation to identification of putative biomarkers.

FIG. 5. Discovery profiling reveals potential metabolic signature. Differential analysis of ASD versus TD using T-test identified metabolites. Computational modeling identified molecular signatures with ˜80% using three methods.

### DETAILED DESCRIPTION OF ILLUSTRATIVE EMBODIMENTS

Autism spectrum disorder (ASD) is a lifelong neurodevelopmental disorder characterized by deficits in social interaction, communication and repetitive or stereotypical behaviors which has recently seen a dramatic increase in prevalence, reaching an estimate of 1 in 50 school-aged children. The diagnosis of ASD at the earliest age possible is important for effective intervention. Earlier diagnosis of children with ASD improves outcomes by providing therapeutic interventions that lead to higher cognitive and social function as well as improved communication, subsequently decreasing the financial and emotional burden on families and society (Dawson et al., 2010, Pediatrics; 125: e17-23; and Ganz, 2007, Arch Pediatr Adolesc Med; 161: 343-349).

The development of metabolic tests for ASD provides an opportunity to identify metabolites that provide etiological information related to inherited biochemical disorders as well the contribution of the gut microbiome, dietary, and environmental factors that create an individual's unique biochemistry. Distinct metabolite based etiological diagnoses allows the heterogeneity of the underlying biology of an individual's ASD to be segregated into distinct subtypes that can lead to more effective therapy. Combining earlier functional and etiological diagnoses can lead to specific treatment options, improved access to treatment, realistic prognosis, and provide patients and families with improved knowledge that may increase positive therapeutic outcomes.

Autism is a heritable disorder and genetic testing can identify underlying genetic changes associated with ASD in 15.8% (Tammimies et al., 2015, JAMA; 314(9):895-903) to 42% (Yuen et al., 2015, Nat Med; 21(2):185-91) of subjects with diagnostic yields believed to be 30% to 40% (Schaefer et al., 2013, Genet Med; 15(5):399-407 and Schaefer et al., 2013 Erratum in: Genet Med; 15(8):669). The prevalent single gene mutations that are often evaluated in ASD gene panels include PTEN (prevalence 8%; Varga et al., 2009, Genet Med; 11(2):111-7), MECP2 (prevalence in females 4%; Zappella et al., 2003, Am J Med Genet B Neuropsychiatr Genet; 119B:102-107), and FMR1 (prevalence 2-6%; Cohen et al., 2005, J Autism Dev Disord; 35(1):103-16) and SHANK3 (prevalence 1%; Moessner et al., 2007, Am J Hum Genet; 81(6):1289-97). Mutations in these genes as well as many other genes associated with ASD are not completely diagnostic for ASD because individuals carrying mutations may not display ASD like behaviors.

Given the complexities of the interactions between genetics and the environment, metabolic profiling can provide an important approach towards a better understanding of ASD and the development of diagnostic tests that aid in individualized treatment decisions. Metabolism based analysis has the potential to identify biomarker profiles derived from an individual's inherited biochemistry as well as capture the interactions of the gut microbiome, dietary, and environmental factors that contribute to the unique metabolic signature of an individual with ASD. Research into the metabolic etiology of this complex disorder has led to a deeper understanding of the underlying causes of ASD and provided avenues for effective intervention. Altered metabolism among individuals with ASD has been observed in numerous endogenous biochemical pathways such as melatonin biosynthesis (Veatch et al., 2015, J Autism Dev Disord; 45(1):100-10), catabolism of branched chain amino acids (Novarino et al., 2012, Science; 338(6105):394-7), fatty acid metabolism (Frye et al., 2013, Transl Psychiatry; 3:e220), methionine transmethylation and transsulfuration pathways (James et al., 2006, Am J Med Genet B Neuropsychiatr Genet; 141B(8):947-56) as well as microbiome related metabolism (Yap 2009; and Ming et al., 2012, J Proteome Res; 11(12):5856-62).

Several syndromes associated with ASD have a metabolic etiology, such as Smith-Lemli-Opitz, and Sanfilippo syndromes, that can be identified through metabolic screening (Zecavati and Spence, 2009, Curr Neurol Neurosci Rep; 9(2):129-36). Multivariate metabolic signatures of ASD have been identified by profiling the blood plasma of ASD and typically developing (TD) children. This research indicated the presence of numerous metabolic perturbations in ASD that could be utilized for biomarker development as well as to further understand the biological basis of ASD (West et al., 2014, PLoS One; 9(11):e112445). Changes in metabolism can be used to stratify individuals with ASD into metabolic subtypes or endophenotypes (James et al., 2006, Am J Med Genet B Neuropsychiatr Genet; 141B(8):947-56; Wang et al., 2010, Autism Res; 3(5):268-72; and Frye et al., 2013, Transl Psychiatry; 3:e220) to describe heterogeneity within the spectrum as well as to inform pharmacological (for example, succinnic semialdehyde dehydrogenase deficiency and vigabatrin) and dietary (for example, biotinidase deficiency and biotin supplementation) interventions that may prevent or ameliorate clinical symptoms. The association of metabolic disorders or altered metabolism within ASD supports the need for metabolic testing of individuals diagnosed with ASD. Discovering altered metabolic functions associated with ASD offers great opportunity for development of molecular diagnostics that can facilitate individualized and efficacious treatment of the disorder that can complement current genetic testing.

Described herein are methods that provide for the identification of metabolic features among individuals with ASD that serve to describe novel, distinct subpopulations within the ASD spectrum, providing value in the diagnosis and individualized treatment of those with ASD.

With the present invention, the metabolite 3-carboxy-4-methyl-5-propyl-2-furanpropanoic acid (CMPF) is identified as a biomarker of a metabolic subtype in ASD (also referred to herein as “an elevated CMPF subset of autism,” “a CMPF-associated subset of autism,” or a “CMPF-subset”). This elevation in CMPF is not associated with an increase in other uremic toxins, such as for example, 3-indoxyl sulfate and/or p-cresol sulfate.

In some aspects, CMPF levels were elevated in this subset to a level of about six times or greater compared to median level in TD individuals. In some aspects, CMPF was elevated at least about 6 times in this subset compared to the median level in TD individuals. In some aspects, CMPF was elevated about 10 times or greater in this subset compared to the median level in TD individuals. In some aspects, CMPF was elevated at least about 10 times in this subset compared to the median level in TD individuals. In some aspects, CMPF was elevated about 6 to about 10 times in this subset compared to the median level in TD individuals.

In some aspects, an elevated CMPF level of about 2 μM or more places an individual in an elevated CNPF subset of autism. In some aspects, a CMPF level of less that about 2 μM excludes the individual from the elevated CMPF subset of autism and/or identifies the individual as TD.

Elevated CMPF levels may be used to identify a CMPF-associated subpopulation within a population of individuals with autism spectrum disorder (ASD).

Elevated CMPF levels provide for the placement of an individual within a CMPF-associated autism subpopulation. Elevated CMPF levels provide for the diagnosis and/or treatment of individuals. In some aspects, the individual has not been previously diagnosed with autism spectrum disorder (ASD). Such an individual may already have been clinically diagnosed with autism spectrum disorder (ASD) and/or is undergoing treatment for autism. In some aspects, one or more metabolites indicative of ASD and/or an ASD subset may be quantified at one or more time points after the initiation of treatment. In some aspects, the level of the one or more metabolites indicative of ASD and/or an ASD subset returns to TD levels after initiation of treatment.

The chemical structure of CMPF (also known as 3-carboxy-4-methyl-5-propyl-2-furanpropanoic acid) is shown below.

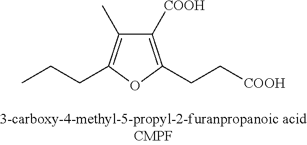 [SMILES: CCCc1oc(CCC(=O)O)c(C(=O)O)c1C]

CMPF is created through the metabolism of dietary furans (esters, acids, tetra alkyl). It is excreted in urine. It accumulates in uremia and is classified as a uremic toxin associated with kidney disease. The serum concentration of in uremic patients aged 40-55 years receiving hemodialysis treatment is 32.3+/−2.7 microg/ml, n=17; mean+/−SEM) (Niwa et al., 1988, Clin Chim Acta; 173(2):127-38; and Sassa et al., 2000, Arch Toxicol; 73(12):649-54). It is bound by albumin, which slows its elimination from plasma, and inhibits tubular secretion in the kidney. CMPF inhibits albumin binding, glutathionine S-transferase, thyroid function, tubular secretion, drug metabolism and insulin synthesis. It is associated with ROS generation, which is linked to autism (Xian et al., 2015, JAMA; 313:1425-1434). CMPF may promote cytotoxicity by inducing oxidative stress in rat neutrophils, rat lymphocytes, human aortic smooth muscle cells, and human proximal tubular cells. CMPF inhibits O-demethylation and glutathione conjugation and glucuronidation. It is incorporated into phospholipids, cholesterol esters, and triglycerides. CMPF is a biomarker associated with gestational diabetes (Prentice et al., 2014, Cell Met; 19:653-666), showing a 7 fold increase to about 100 μM. A diet with fish oil supplementation can demonstrate a 3 fold increase an active tuberculosis infection can demonstrate a 2 fold decrease, and a “healthy diet” can demonstrate a 2.5 fold increase. CMPF inhibits OAT3 transporters, inhibiting OAT3 efflux from brain to blood, leading to the accumulation of toxic neurotransmitter metabolites in the brain (Ohtsuki et al., 2002, J Neurochem; 83:57-66).

In some aspects, an elevated CMPF subset of autism represents about 5% to about 20%, about 5% to about 15%, about 8% to about 14%, less than about 20%, less than about 15%, about 14%, or about 12% of the general ASD population.

The present invention includes methods of measuring the levels of the metabolite 3-carboxy-4-methyl-5-propyl-2-furanpropanoic acid (CMPF) in a biosample obtained from a human subject. One or more additional metabolites, including, but not limited to, any of those described here, may also be measured.

For example, one or more non-CMPF markers of uremia, including, but not limited to, 3-indoxyl sulfate and/or p-cresol sulfate, may be measured to determine that the individual is not suffering from uremia.

For example, one or more 2-omega fatty acid metabolites, including, but are not limited to, for example, fish oil, EPA, and/or DAH may be measured to determine if the subject has been consuming a 2-omega fatty acid.

With the present invention, additional metabolites indicative of ASD have been identified. This includes, for example, any one or more of the metabolites described in examples included herewith. For example, additional metabolites indicative of ASD may include any one or more of the following metabolites: 3-hydroxy-3-methylbutyric acid, 3-methyl-2-oxovaleric acid, salicylic acid, gentisic acid, a CMPF-related metabolite, DHEA sulfate, pregnenolone sulfate, LysoPE(22:6), glycine, 1-alanine, sarcosine, and/or proline betaine.

In some aspects, 3-hydroxy-3-methylbutyric acid of a fold change range of ASD/TD of about 0 to about 0.84, 3-methyl-2-oxovaleric acid of a fold change range of ASD/TD of about 0 to about 0.87, salicylic acid of a fold change range of ASD/TD of about 0 to about 0.77, gentisic acid of fold change range of a ASD/TD of about 0 to about 0.71, and/or proline betaine of a fold change range of ASD/TD of about 0 to about 0.72 is indicative of autism.

In some aspects, the CMPF-related metabolite of a fold change range of TD/ASD of about 0 to about 0.12, DHEA sulfate of a fold change range of TD/ASD of about 0 to about 0.38, pregnenolone sulfate of a fold change range of TD/ASD of about 0 to about 0.58, LysoPE(22:6) of a fold change range of TD/ASD of about 0 to about 0.78, glycine of a fold change range of TD/ASD of about 0 to about 0.75, 1-alanine of a fold change range of TD/ASD of about 0 to about 0.86, sacrosine of a fold change range of TD/ASD of about 0 to about 0.85 is indicative of autism.

In some aspects, 3-hydroxy-3-methylbutyric acid of a fold change range of ASD/TD is less than 0.84, 3-methyl-2-oxovaleric acid of a fold change range of ASD/TD of less than 0.87, salicylic acid of a fold change range of ASD/TD of less than 0.77, gentisic acid of a fold change range of ASD/TD of less than 0.71, the CMPF-related metabolite of a fold change range of ASD/TD of greater than about 8.43, DHEA sulfate of a fold change range of ASD/TD of greater than about 2.63, pregnenolone sulfate of a fold change range of ASD/TD of greater than about 1.71, LysoPE(22:6) of a fold change range of ASD/TD of greater than about 1.38, glycine of a fold change range of ASD/TD of greater than about 1.34, 1-alanine of a fold change range of ASD/TD of greater than about 1.17, sacrosine of a fold change range of ASD/TD of greater than about 1.17, and/or proline betaine of a fold change range of ASD/TD of greater than about 0.72 is indicative of autism.

In some aspects, a measurement of 3-hydroxy-3-methylbutyric acid is less than 0.84 times the TD average or 1/0.84 times greater than the TD average, the measurement of 3-methyl-2-oxovaleric acid is less than 0.87 times the TD average or 1/0.87 times greater than the TD average, salicylic acid is less than 0.77 the TD average or 1/0.77 greater than the TD average, gentisic acid is less than 0.71 times the TD average or 1/0.71 times greater than the TD average, the CMPF-related metabolite is less than 8.43 times the TD average or 1/8.43 times greater than the TD average, DHEA sulfate is less than 2.63 times the TD average or 1/2.63 times greater than the TD average, pregnenolone sulfate is less than 1.71 times the TD average or 1/1.71 times greater than the TD average, LysoPE(22:6) is less than 1.38 times the TD average or 1/1.38 times greater than the TD average, glycine is less than 1.34 times the TD average or 1/1.34 times greater than the TD average, 1-alanine is less than 1.17 times the TD average or 1/1.17 times greater than the TD average, sacrosine is less than 1.17 times the TD average or 1/1.17 times greater than the TD average, and/or proline betaine is less than 0.72 times the TD average or 1/0.72 times greater than the TD average is indicative of autism.

In some aspects, as used herein, “a fold change range of ASD/TD of” may also be referred to as “a fold change range of an ASD subject in a ratio with the population mean TD of” and “a fold change range of TD/ASD” may also be referred to as “a fold change range of the population mean TD in a ratio with an ASD subject.”

Biosamples may be obtained from any of a variety of mammalian subjects. In preferred embodiments, a biosample is from a human subject. A biosample may be from an individual clinically diagnosed with ASD. ASD may be diagnosed by any of a variety of well-known clinical criteria. For example, diagnosis of autism spectrum disorder may be based on the DSM-IV criteria determined by an experienced neuropsychologist and/or the Autism Diagnostic Observation Schedule-Generic (ADOS-G) which provides observation of a child's communication, reciprocal social interaction, and stereotyped behavior including an algorithm with cutoffs for autism and autism spectrum disorders.

A biosample may be from an individual determined to be at some risk for ASD (for example by family history) with little or no current ASD symptoms. A biosample may be a suitable reference or control sample from an individual not suffering from ASD with or without a family history of ASD. In some aspects, a plurality of samples is obtained from a population, for example, a population of individuals with ASD, at risk for ASD, or normal, typically developing (TD) individuals. In some aspects, a biosample may be from an individual determined to be developmentally delayed (DD) including with impairment in physical learning, language, and/or behavior, an individual with a neuro-developmental disorder, or a typically dedeveloping individual (TD).

A biosample may be from an adult subject. A biosample may be from an adult. A biosample may be from a teenager. A biosample may be from a child, for example, a child that is under about 6 years of age, under about 5 years of age, under about 4 years of age, under about 3 years of age, under about 2 years of age, under 18 months of age, or under about 1 year of age, about 1 to about 6 years of age, about 1 to about 5 years of age, about 1 to about 4 years of age, about 1 to about 2 years of age, about 2 to about 6 years of age, about 2 to about 4 years of age, or about 4 to about 6 years of age. A biosample may be from a phenotypic subpopulation of autism subjects, such as, for example, high functioning autism (HFA) or low functioning autism (LFA). A sample may be form a typically developing (TD) individual. A biosample may be from a developmentally delayed (DD) individual demonstrating impairment in physical learning, language, and/or behavior. A biosample may be from an individual with a neuro-developmental disorder developmentally.

In accordance with the methods disclosed herein, any type of biological sample that originates from anywhere within the body of a subject may be tested, including, but not limited to, blood (including, but no limited to serum or plasma), cerebrospinal fluid (CSF), pleural fluid, urine, stool, sweat, tears, breath condensate, saliva, vitreous humour, a tissue sample, amniotic fluid, a chorionic villus sampling, brain tissue, a biopsy of any solid tissue including tumor, adjacent normal, smooth and skeletal muscle, adipose tissue, liver, skin, hair, brain, kidney, pancreas, lung, colon, stomach, or the like may be used. A blood sample may include, for example, a whole blood sample, a blood serum sample, a blood plasma sample, or other blood components, such as, for example, a subfraction or an isolated cellular subpopulation of whole blood. In some aspects a biosample may be a cellular membrane preparation. A sample may be from a live subject. In some applications, samples may be collected post mortem.

When a blood sample is drawn from a subject, it can be processed in any of many known ways. The range of processing can be from little to none (such as, for example, frozen whole blood) or as complex as the isolation of a particular cell type. Common and routine procedures include the preparation of either serum or plasma from whole blood. All blood sample processing methods, including spotting of blood samples onto solid-phase supports, such as filter paper or other immobile materials, are contemplated by the present invention.

With the preparation of samples for analysis, metabolites may be extracted from their biological source using any number of extraction/clean-up procedures that are typically used in quantitative analytical chemistry.

In some aspects, a method for diagnosing and/or subtyping autism based on identification and/or quantification of one or more signature metabolites as described herein may further include the identification and/or quantification of one or more additional known markers of autism. For example, one or more of the markers and/or methodologies for their identification and/or quantification as described in US Patent Application 20120190055 (“Molecule Biomarkers of Autism”), which is hereby incorporated by reference in its entirety, may be used. One or more of the markers and/or the methodologies for their identification and/or quantification as described in U.S. Pat. No. 8,273,575 (“Methods for the diagnosis, risk assessment, and monitoring of autism spectrum disorders”), which is hereby incorporated by reference in its entirety, may be used. In some aspects, the nucleic acids from a biological sample may be analyzed to determine the genotype and/or expression of genes associated with or relevant to autism.

In some aspects, a method of diagnosing and/or subtyping autism may include assaying a biosample from the subject for one or a plurality of small molecule metabolites and quantifying the amount of one or more of the metabolites selected from 3-hydroxy-3-methylbutyric acid, 3-methyl-2-oxovaleric acid, salicylic acid, gentisic acid, a CMPF-related metabolite, DHEA sulfate, pregnenolone sulfate, LysoPE(22:6), glycine, 1-alanine, sacrosine, and/or proline betaine.

In some aspects, 3-hydroxy-3-methylbutyric acid of a fold change range of ASD/TD of about 0 to about 0.84, 3-methyl-2-oxovaleric acid of a fold change range of ASD/TD of about 0 to about 0.87, salicylic acid of a fold change range of ASD/TD of about 0 to about 0.77, gentisic acid of a fold change range of ASD/TD of about 0 to about 0.71, and/or proline betaine of a fold change range of ASD/TD of about 0 to about 0.72 places the individual in the autism subpopulation.

In some aspects, the CMPF-related metabolite of a fold change range of TD/ASD of about 0 to about 0.12, DHEA sulfate of a fold change range of TD/ASD of about 0 to about 0.38, pregnenolone sulfate of a fold change range of TD/ASD of about 0 to about 0.58, LysoPE(22:6) of a fold change range of TD/ASD of about 0 to about 0.78, glycine of a fold change range of TD/ASD of about 0 to about 0.75, 1-alanine of a fold change range of TD/ASD of about 0 to about 0.86, sacrosine of a fold change range of TD/ASD of about 0 to about 0.85 places the individual in the autism subpopulation.

In some aspects, 3-hydroxy-3-methylbutyric acid of a fold change range of ASD/TD is less than 0.84, 3-methyl-2-oxovaleric acid of a fold change range of ASD/TD of less than 0.87, salicylic acid of a fold change range of ASD/TD of less than 0.77, gentisic acid of a fold change range of ASD/TD of less than 0.71, the CMPF-related metabolite of a fold change range of ASD/TD of greater than about 8.43, DHEA sulfate of a fold change range of ASD/TD of greater than about 2.63, pregnenolone sulfate of a fold change range of ASD/TD of greater than about 1.71, LysoPE(22:6) of a fold change range of ASD/TD of greater than about 1.38, glycine of a fold change range of ASD/TD of greater than about 1.34, 1-alanine of a fold change range of ASD/TD of greater than about 1.17, sacrosine of a fold change range of ASD/TD of greater than about 1.17, and/or proline betaine of a fold change range of ASD/TD of greater than about 0.72 places the individual in the autism subpopulation.

In some aspects, a measurement of 3-hydroxy-3-methylbutyric acid is less than 0.84 times the TD average or 1/0.84 times greater than the TD average, the measurement of 3-methyl-2-oxovaleric acid is less than 0.87 times the TD average or 1/0.87 times greater than the TD average, salicylic acid is less than 0.77 the TD average or 1/0.77 greater than the TD average, gentisic acid is less than 0.71 times the TD average or 1/0.71 times greater than the TD average, the CMPF-related metabolite is less than 8.43 times the TD average or 1/8.43 times greater than the TD average, DHEA sulfate is less than 2.63 times the TD average or 1/2.63 times greater than the TD average, pregnenolone sulfate is less than 1.71 times the TD average or 1/1.71 times greater than the TD average, LysoPE(22:6) is less than 1.38 times the TD average or 1/1.38 times greater than the TD average, glycine is less than 1.34 times the TD average or 1/1.34 times greater than the TD average, 1-alanine is less than 1.17 times the TD average or 1/1.17 times greater than the TD average, sacrosine is less than 1.17 times the TD average or 1/1.17 times greater than the TD average, and/or proline betaine is less than 0.72 times the TD average or 1/0.72 times greater than the TD average places the individual in the autism subpopulation.

In some aspects, a method of diagnosing and/or subtyping autism may include assaying a biosample from the subject for one or a plurality of small molecule metabolites and quantifying the amount of one or more of the various small molecule metabolites described in International Application No.: PCT/US2014/045397 (“Biomarkers of Autism Spectrum Disorder”), which is hereby incorporated by reference in its entirety. Such metabolites may include one or more of the metabolites listed in Table 5 International Application No.: PCT/US2014/045397. For example, any one or more of the metabolites, any two or more metabolites, any three or more metabolites, any four or more metabolites, any five or more metabolites, any six or more metabolites, any seven or more metabolites, any eight or more metabolites, any nine or more metabolites, any ten or more metabolites, any eleven or more metabolites, any twelve or more metabolites, any thirteen or more metabolites, any fourteen or more metabolites, any fifteen or more metabolites, any sixteen or more metabolites, any seventeen or more metabolites, any eighteen or more metabolites, any nineteen or more metabolites, any twenty or more metabolites, or twenty one metabolites selected from those listed in Table 5 of International Application No.: PCT/US2014/045397.

In some aspects, a method of diagnosing and/or subtyping autism may include assaying a biosample from the subject for one or a plurality of small molecule metabolites and quantifying the amount of one or more of the 179 small molecule metabolites listed in Table 6 of International Application No.: PCT/US2014/045397 (“Biomarkers of Autism Spectrum Disorder”). For example, such metabolites may include any one or more of the metabolites, any two or more metabolites, any three or more metabolites, any four or more metabolites, any five or more metabolites, any six or more metabolites, any seven or more metabolites, any eight or more metabolites, any nine or more metabolites, any ten or more metabolites, any eleven or more metabolites, any twelve or more metabolites, any thirteen or more metabolites, any fourteen or more metabolites, any fifteen or more metabolites, any sixteen or more metabolites, any seventeen or more metabolites, any eighteen or more metabolites, any nineteen or more metabolites, any twenty or more metabolites, or twenty one metabolites of homocitrulline, 2-hydroxyvaleric acid, cystine, aspartic acid, isoleucine, creatinine, serine, 4-hydroxyphenyllactic acid, citric acid, glutamic acid, lactic acid, DHEA sulfate, glutaric acid, 5-hydroxynorvaline, heptadecanoic acid, 5-aminovaleric acid lactam, succinic acid, myristic acid, 2-hydroxyvaleric acid, methylhexadecanoic acid, and/or 3-aminoisobutyric acid. These are described in more detail in International Application No.: PCT/US2014/045397 (“Biomarkers of Autism Spectrum Disorder”).

The present invention includes a metabolomic signature for a subset of autism as described herein. In some aspects, the metabolomic signature includes 3-carboxy-4-methyl-5-propyl-2-furanpropanoic acid (CMPF). In some aspects, the concentration of CMPF is at least about six times or greater than the median level in TD individuals and/or at least about 2 μM or greater. In some aspects, a metabolomic signature for a subset of autism may further include one or more of 3-hydroxy-3-methylbutyric acid, 3-methyl-2-oxovaleric acid, salicylic acid, gentisic acid, a CMPF-related metabolite, DHEA sulfate, pregnenolone sulfate, LysoPE(22:6), glycine, 1-alanine, sacrosine, and/or proline betaine.

A metabolomic signature for autism may further include any one or more of, any one or more of the metabolites, any two or more metabolites, any three or more metabolites, any four or more metabolites, any five or more metabolites, any six or more metabolites, any seven or more metabolites, any eight or more metabolites, any nine or more metabolites, any ten or more metabolites, any eleven or more metabolites, any twelve or more metabolites, any thirteen or more metabolites, any fourteen or more metabolites, any fifteen or more metabolites, any sixteen or more metabolites, any seventeen or more metabolites, any eighteen or more metabolites, any nineteen or more metabolites, any twenty or more metabolites, or twenty one or more metabolites, any twenty two or more metabolites, any twenty three or more metabolites, any twenty four or more metabolites, any twenty five or more metabolites, and/or twenty six metabolites of 2-aminooctanoic acid, acesulfame, ADMA, choline, CMPF, cysteine, cystine, DHEA sulfate (DHEAS), glycine, glycocholic acid, hypoxanthine, indoleacrylic acid, indoxyl sulfate, LysoPC(16:1(9Z)), LysoPE(0:0/18:1(9Z)), LysoPE(22:6(4Z,7Z,10Z,13Z,16Z,19Z)/0:0), LysoPE(22:6(4Z,7Z,10Z,13Z,16Z,19Z)/0:0), methionine, p-cresol sulfate, phenylalanine, phenyllactic acid, proline, serotonin, tryptophan, uric acid, and/or valine. These are described in more detail in International Application No.: PCT/US2014/045397 (“Biomarkers of Autism Spectrum Disorder”), which is hereby incorporated by reference in its entirety.

Metabolic biomarkers may be identified by their unique molecular mass and consistency, thus the actual identity of the underlying compound that corresponds to the biomarker is not required for the practice of this invention.

This may be measured as an average abundance ratio relative to a normal control. In some aspects, an average abundance ratio of other than about 1 may be indicative of autism. For example, an average abundance ratio of greater than about 1 (for example, including, but not limited to, about 1.01, about 1.02, about 1.03, about 1.04, about 1.05, about 1.06, about 1.07, about 1.08, about 1.09, about 1.1, about 1.11, about 1.12, about 1.13, about 1.14, about 1.15, about 1.16, about 1.17, about 1.18, about 1.19, about 1.2, about 1.21, about 1.22, about 1.23, about 1.24, about 1.25, about 1.26, about 1.27, about 1.28, about 1.29, about 1.3, about 1.31, about 1.32, about 1.33, about 1.34, about 1.35, about 1.36, about 1.37, about 1.38, about 1.39, about 1.4, about 1.41, about 1.42, about 1.43, about 1.44, about 1.45, about 1.46, about 1.47, about 1.48, about 1.49, or about 1.5) may be indicative of autism. In some aspects, an average abundance ratio of less than about 1 (for example, including, but not limited to, about 0.99, about 0.98, about 0.97, about 0.96, about 0.95, about 0.94, about 0.93, about 0.92, about 0.91, about 0.9, about 0.89, about 0.88, about 0.87, about 0.86, about 0.85, about 0.84, about 0.83, about 0.82, about 0.81, about 0.8, about 0.79, about 0.78, about 0.77, about 0.76, about 0.75, about 0.74, about 0.73, about 0.72, about 0.71, about 0.7, about 0.69, about 0.68, about 0.67, about 0.66, about 0.65, about 0.64, about 0.63, about 0.62, about 0.61, about 0.6, about 0.59, about 0.58, about 0.57, about 0.56, about 0.55, about 0.54, about 0.53, about 0.52, about 0.51, or about 0.5) may be indicative of autism.

The metabolic markers and signatures described herein may be utilized in tests, assays, methods, kits for diagnosing, predicting, modulating, or monitoring ASD, including ongoing assessment, monitoring, susceptibility assessment, carrier testing and prenatal diagnosis.

Metabolic biomarkers may be identified by their unique molecular mass and consistency, thus the actual identity of the underlying compound that corresponds to the biomarker is not required for the practice of this invention. Biomarkers may be identified using, for example, Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, tandem mass spectrometry (e.g., MS/MS, MS/MS/MS, ESI-MS/MS etc.), secondary ion mass spectrometry (SIMS), and/or ion mobility spectrometry (e.g. GC-IMS, IMS-MS, LC-IMS, LC-IMS-MS etc.).

Metabolites as set forth herein can be detected using any of the methods described herein. Metabolites, as set forth herein, can be detected using alternative spectrometry methods or other methods known in the art, in addition to any of those described herein.

In some aspects, the determination of a metabolite may be by a methodology other than a physical separation method, such as for example, a colorimetric, enzymatic, immunological methodology, and gene expression analysis, including, for example, real-time PCR, RT-PCR, Northern analysis, and in situ hybridization.

In some aspects, the quantification of one or more small molecule metabolites of a metabolic signature of autism may be assayed using a physical separation method, such as, for example, one or more methodologies selected from gas chromatography mass spectrometry (GCMS), C8 liquid chromatography coupled to electrospray ionization in positive ion polarity (C8pos), C8 liquid chromatography coupled to electrospray ionization in negative ion polarity (C8neg), hydrophilic interaction liquid chromatography coupled to electrospray ionization in positive ion polarity (HILICpos), and/or hydrophilic interaction liquid chromatography coupled to electrospray ionization in negative ion polarity (HILICneg).

With any of the methods described herein, any combination of one or more gas chromatography-mass spectrometry (GC-MS) methodologies and/or one or more liquid chromatography-high resolution mass spectrometry (LC-HRMS) methodologies may be used. In some aspects, a GC-MS method may be targeted. In some aspects, a LC-HRMS method may be untargeted. Subsequently, in some embodiments, tandem mass spectrometry (MS-MS) methods may be employed for the structural confirmation of metabolites. LC-HRMS methodologies may include C8 chromatography and/or Hydrophilic Interaction Liquid Chromatography (HILIC) chromatography. Either of C8 chromatography or HILIC chromatography may be coupled to electrospray ionization in both positive and negative ion polarities, resulting in multiple data acquisitions per sample.

In some aspects of the methods described herein, concentrations of one or more metabolites, including, but not limited to CMPF, may be determined using C18 (reverse phase) LC coupled with a triple quadrupole (QqQ) MS using electrospray ionization in the positive ion mode with analyte detection in the multiple reaction monitoring (MRM) mode. This may include a stable label internal standard and CMPF concentrations are measured distributed over a linear range of 0.05 to 100 μM.

The present invention includes a kit for identifying and/or measuring one or more metabolites associated with a subset of ASD. In some aspects, the kit may be for the determination of a metabolite by a physical separation method. In some aspects, the kit may be for the determination of a metabolite by a methodology other than a physical separation method, such as for example, a colorimetric, enzymatic, immunological methodology. In some aspects an assay kit may also include one or more appropriate negative controls and/or positive controls. Kits of the present invention may include other reagents such as buffers and solutions needed to practice the invention are also included. Optionally associated with such container(s) can be a notice or printed instructions. As used herein, the phrase “packaging material” refers to one or more physical structures used to house the contents of the kit. The packaging material is constructed by well-known methods, preferably to provide a sterile, contaminant-free environment. As used herein, the term “package” refers to a solid matrix or material such as glass, plastic, paper, foil, and the like. Kits of the present invention may also include instructions for use. Instructions for use typically include a tangible expression describing the reagent concentration or at least one assay method parameter, such as the relative amounts of reagent and sample to be admixed, maintenance time periods for reagent/sample admixtures, temperature, buffer conditions, and the like. In some aspects, a kit may be a packaged combination including the basic elements of a first container including, in solid form, a specific set of one or more purified metabolites, as described herein, and a second container including a physiologically suitable buffer for resuspending or dissolving the specific subset of purified metabolites. Such a kit may be used by a medical specialist to determine whether or not a subject is at risk for ASD. Appropriate therapeutic intervention may be prescribed or initiated upon the determination of a risk of ASD. One or more of the metabolites described herein may be present in a kit.

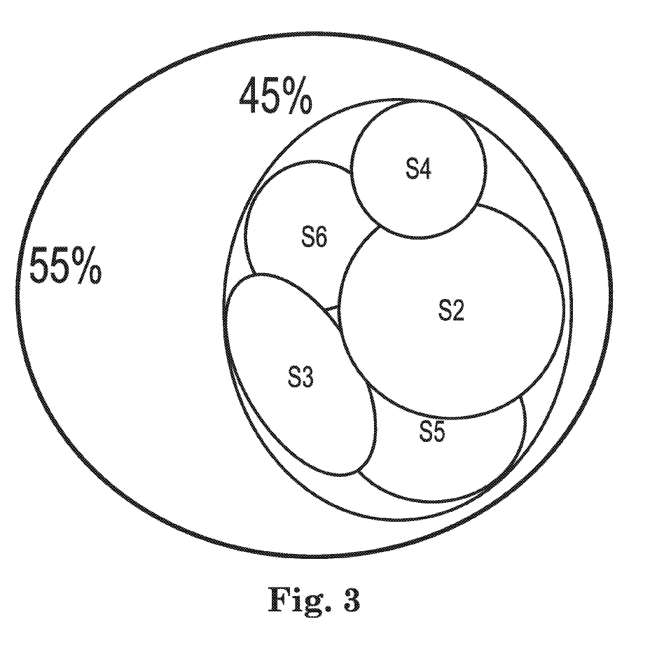

The present invention includes a simple abundance threshold method for identifying one or more metabolites identifying a subpopulation within a population of individuals with autism spectrum disorder (ASD), or a method of identifying a subpopulation within a population of individuals with ASD. This method is used in Example 3 to obtain the Venn diagram of autism subsets shown in FIG. 3. This method may include:

measuring the levels of one or more features (for example, metabolites, putative-metabolites, unknown metabolites, proteins, and/or RNA) in two populations;

determining in one population an optimal upper threshold for each feature, wherein the upper threshold represents a level of the feature wherein all except 1% of subjects with a TD diagnosis has a level of feature which is below the threshold;

counting the number of subjects with a diagnosis of ASD which have feature levels above the upper threshold and saving as a hypothetical diagnostic features where this count is above about 6% of the total number of ASD subjects;

repeating the above steps for all features wherein a lower threshold is used which represents a level for which all but one TD subject has levels above the lower threshold;

saving as a hypothetical diagnostic every feature for which about 6% or greater of ASD subjects have feature levels below the lower threshold;

creating a multitude of feature ratios for each hypothetical diagnostic by dividing the level determined for each subject by a set of normalizing features (this set can be all features or a selected set of features or determined metabolites or putative metabolites);

determining ratio optimal thresholds for each feature;

determining the percent of ASD subjects which have ratios above or below the thresholds, wherein ratios which distinguish the greatest number of ASD subjects are saved as diagnostic ratio;

using a second population of subjects wherein the age, demographics and collection conditions (for example fasted or non-fasted) can be the same or different from the first study; and

determining the performance of each diagnostic ratio using the same optimal threshold which was determined in the first population of subjects;

wherein ratio diagnostics which perform with greater than about 90% specificity and about 6% sensitivity (or any performance requirements one sees fit) reveal features which define a subtype in ASD.

In some aspects, one or more features may include a confirmed metabolite. In some aspects, one or more features may include a putative metabolite as determined by matching a library of features associated with known metabolites on mass and retention time.

In some aspects, the first population and the second population includes subsets of a single study. In some aspects, the first population and the second population include two independent studies.

In some aspects, feature levels and/or and diagnostic ratios are determined by the same or different techniques. In some aspects, feature levels and/or and diagnostic ratios are determined using mass spectrometry. In some aspects, mass spectrometry includes gas chromatography mass spectrometry (GC-MS), and liquid chromatography mass spectrometry (e.g. LC-MS, LC-MS-MS, LC-MRM, LC-SIM, LC-SRM) using reverse phase liquid chromatography coupled to electrospray ionization in positive ion polarity (C8pos), reverse phase liquid chromatography coupled to electrospray ionization in negative ion polarity (C8neg), hydrophilic interaction liquid chromatography coupled to electrospray ionization in positive ion polarity (HILICpos), and/or hydrophilic interaction liquid chromatography coupled to electrospray ionization in negative ion polarity (HILICneg).

In some aspects, mass spectrometry includes C18 (reverse phase) LC coupled with a triple quadrupole (QqQ) MS using electrospray ionization in the positive ion mode with analyte detection in the multiple reaction monitoring (MRM) mode including a stable label internal standard wherein CMPF concentrations are measured distributed over a linear range of 0.05 to 100 μM.

In some aspects, selected features used in the denominator of the diagnostic ratio are selected to be non-complementary to the numerator or hypothetical diagnostic feature (i.e. is not itself a hypothetical diagnostic feature). In some aspects, the selected features used in the denominator are determined to be hypothetical diagnostics which when used in the denominator improve the diagnostic performance of the ratio (i.e. complementary to the feature in the numerator).

In some aspects, one or more metabolite(s) used for the denominator is a spiked-in agent.

In some aspects, the optimal upper threshold is defined as the level of feature for which all ASD or DD subjects are below the threshold and the optimal lower threshold is defined as the level of feature for all ASD or DD subjects which is above the threshold.

In some aspects, the optimal lower and upper thresholds are:

based on measures of dispersion population (for example, variance, IQR, MAD, CV, standard deviation, standard error and/or other statistical means) of the mean or median of the non-ASD;

based on measures of dispersion population (for example, variance, IQR, MAD, CV, standard deviation, standard error and/or other statistical means) of the mean or median of the ASD population;

based on the upper and lower quantiles of the non-ASD population;

based on the upper and lower quantiles of the ASD population;

based on a measure of statistical distance of the subjects with ASD to non-ASD subjects;

based on a standard score or standardized variable of non-ASD subjects;

based on a standard score or standardized variable of ASD subjects; or based on ROC AUC.

In some aspects, the minimum percentage sensitivity required for the determination of a hypothetical diagnostic includes about 3%, about 4%, about 5%, about 7%, about 8%, about 9%, about 10%, about 11.%, about 12%, about 13%, about 14%, about 15%, about 16%, about 17%, about 18%, about 19%, or about 20%, rather than about 6%; the ratio diagnostics perform with greater than at least about 95% specificity, at least about 96% specificity, at least about 97% specificity, at least about 98% specificity, or at least about 99% specificity; and/or the ratio diagnostics perform with at least about 75% specificity, at least about 80% specificity, at least about 85% specificity, at least about 86% specificity, at least about 87% specificity, at least about 88% specificity, or at least about 89% specificity, rather than greater than about 90% specificity.

As used herein, a “training set” is a set of data used in various areas of information science to discover potentially predictive relationships. Training sets are used in artificial intelligence, machine learning, genetic programming, intelligent systems, and statistics. In all these fields, a training set has much the same role and is often used in conjunction with a test set.

As used herein, a “test set” is a set of data used in various areas of information science to assess the strength and utility of a predictive relationship. Test sets are used in artificial intelligence, machine learning, genetic programming, intelligent systems, and statistics. In all these fields, a test set has much the same role.

Data collected during analysis may be quantified for one or more than one metabolite. Quantifying data may be obtained by measuring the levels or intensities of specific metabolites present in a sample. The quantifying data may be compared to corresponding data from one or more than one reference sample. A “reference sample” is any suitable reference sample for the particular disease state. For example, a reference sample may be a sample from a control individual, i.e., a person not suffering from ASD with or without a family history of ASD (also referred to herein as a “typically developing individual” (TD), or “normal” counterpart. A reference sample may also be a sample obtained from a patient clinically diagnosed with ASD. As would be understood by a person of skill in the art, more than one reference sample may be used for comparison to the quantifying data.

As used herein, the term “metabolite” or “cellular metabolite” refers to specific small molecules, the levels or intensities of which are measured in a sample, and that may be used as markers to diagnose a disease state. As used herein, the term “feature” refers to a single small metabolite, or a fragment of a metabolite. Metabolites include, but are not limited to, sugars, organic acids, amino acids, fatty acids, hormones, vitamins, acids, bases, lipids, glycosides, amines, oximes, esters, dipeptides, tripeptides, cholesterols, oxysterols, glycerols, steroids, oligopeptides (less than about 100 amino acids in length), as well as ionic fragments thereof. In some aspects, metabolites are less than about 3000 Daltons in molecular weight. In some aspects, metabolites are less than about 1500 Daltons in molecular weight. In some aspects, metabolites are from about 10 to about 3000 Daltons in molecular weight. In some aspects, metabolites are from about 50 to about 3000 Daltons in molecular weight. In some aspects, metabolites are from about 10 Daltons to about 1500 Dalton in molecular weight. In some aspects, metabolites are from about 50 Daltons to about 1500 Dalton in molecular weight.

As used herein, the term “biomarker” or “metabolic biomarker” refers to metabolites that exhibit statistically significant alterations between diseased and controls.

The terms “metabolic signature” and “biomarker profile” as used herein refer to one or a plurality of metabolites identified by the inventive methods. A metabolic signature of a subset of autism is a population of cellular metabolites that are significantly altered in a subset of autistic patient biofluids, providing a molecular fingerprint of autism spectral disorders. Such a metabolic signature of an autism subset may be used to diagnose autism in an individual and/or treatment of autism.

A computer may be used for statistical analysis. Data for statistical analysis can be extracted from chromatograms (spectra of mass signals) using softwares for statistical methods known in the art. “Statistics” is the science of making effective use of numerical data relating to groups of individuals or experiments. Methods for statistical analysis are well-known in the art. In one embodiment a computer is used for statistical analysis. In one embodiment, the Agilent MassProfiler or MassProfilerProfessional software is used for statistical analysis. In another embodiment, the Agilent MassHunter software Qual software is used for statistical analysis. In other embodiments, alternative statistical analysis methods can be used. Such other statistical methods include the Analysis of Variance (ANOVA) test, Chi-square test, Correlation test, Factor analysis test, Mann-Whitney U test, Mean square weighted derivation (MSWD), Pearson product-moment correlation coefficient, Regression analysis, Spearman's rank correlation coefficient, Student's T test, Welch's T-test, Tukey's test, and Time series analysis. In different embodiments signals from mass spectrometry can be transformed in different ways to improve the performance of the method. Either individual signals or summaries of the distributions of signals (such as mean, median or variance) can be so transformed. Possible transformations include taking the logarithm, taking some positive or negative power, for example the square root or inverse, or taking the arcsin. In different embodiments, statistical classification algorithms are used to create a classification model in order to predict autism and non-autism. Machine learning-based classifiers have been applied in various fields such as machine perception, medical diagnosis, bioinformatics, brain-machine interfaces, classifying DNA sequences, and object recognition in computer vision. Learning-based classifiers have proven to be highly efficient in solving some biological problems.

“Sensitivity” and “specificity” are statistical measures of the performance of a binary classification test. Sensitivity measures the proportion of actual positives which are correctly identified as such (e.g. the percentage of sick people who are correctly identified as having the condition). Specificity measures the proportion of negatives which are correctly identified (e.g. the percentage of healthy people who are correctly identified as not having the condition). These two measures are closely related to the concepts of type I and type II errors. A theoretical, optimal prediction can achieve 100% sensitivity (i.e. predict all people from the sick group as sick) and 100% specificity (i.e. not predict anyone from the healthy group as sick). A specificity of 100% means that the test recognizes all actual negatives—for example, in a test for a certain disease, all disease free people will be recognized as disease free. A sensitivity of 100% means that the test recognizes all actual positives—for example, all sick people are recognized as being ill. Thus, in contrast to a high specificity test, negative results in a high sensitivity test are used to rule out the disease. A positive result in a high specificity test can confirm the presence of disease. However, from a theoretical point of view, a 100%-specific test standard can also be ascribed to a ‘bogus’ test kit whereby the test simply always indicates negative. Therefore the specificity alone does not tell us how well the test recognizes positive cases. Knowledge of sensitivity is also required. For any test, there is usually a trade-off between the measures. For example, in a diagnostic assay in which one is testing for people who have a certain condition, the assay may be set to overlook a certain percentage of sick people who are correctly identified as having the condition (low specificity), in order to reduce the risk of missing the percentage of healthy people who are correctly identified as not having the condition (high sensitivity). Eliminating the systematic error improves accuracy but does not change precision. This trade-off can be represented graphically using a receiver operating characteristic (ROC) curve.

The “accuracy” of a measurement system is the degree of closeness of measurements of a quantity to its actual (true) value. The “precision” of a measurement system, also called reproducibility or repeatability, is the degree to which repeated measurements under unchanged conditions show the same results. Although the two words can be synonymous in colloquial use, they are deliberately contrasted in the context of the scientific method. A measurement system can be accurate but not precise, precise but not accurate, neither, or both. For example, if an experiment contains a systematic error, then increasing the sample size generally increases precision but does not improve accuracy.

The term “predictability” (also called banality) is the degree to which a correct prediction or forecast of a system's state can be made either qualitatively or quantitatively. Perfect predictability implies strict determinism, but lack of predictability does not necessarily imply lack of determinism. Limitations on predictability could be caused by factors such as a lack of information or excessive complexity.

In some embodiments, the invention discloses a method for diagnosing autism with at least about 80% accuracy, at least about 81% accuracy, at least about 82% accuracy, at least about 83% accuracy, at least about 84% accuracy, at least about 85% accuracy, at least about 86% accuracy, at least about 87% accuracy, at least about 88% accuracy, at least about 89% accuracy, at least about 90% accuracy, at least about 91% accuracy, at least about 92% accuracy, at least about 93% accuracy, at least about 94% accuracy, at least about 95% accuracy, at least about 96% accuracy, at least about 97% accuracy, at least about 98% accuracy, or at least about 99% accuracy.

In some embodiments, the invention discloses a method for diagnosing autism with at least about 80% sensitivity, at least about 81% sensitivity, at least about 82% sensitivity, at least about 83% sensitivity, at least about 84% sensitivity, at least about 85% sensitivity, at least about 86% sensitivity, at least about 87% sensitivity, at least about 88% sensitivity, at least about 89% sensitivity, at least about 90% sensitivity, at least about 91% sensitivity, at least about 92% sensitivity, at least about 93% sensitivity, at least about 94% sensitivity, at least about 95% sensitivity, at least about 96% sensitivity, at least about 97% sensitivity, at least about 98% sensitivity, or at least about 99% sensitivity.

In some embodiments, the invention discloses a method for diagnosing autism with at least about 75% specificity, at least about 80% specificity, at least about 81% specificity, at least about 82% specificity, at least about 83% specificity, at least about 84% specificity, at least about 85% specificity, at least about 86% specificity, at least about 87% specificity, at least about 88% specificity, at least about 89% specificity, at least about 90% specificity, at least about 91% specificity, at least about 92% specificity, at least about 93% specificity, at least about 94% specificity, at least about 95% specificity, at least about 96% specificity, at least about 97% specificity, at least about 98% specificity, or at least about 99% specificity.

In some embodiments, the invention discloses a method for diagnosing autism with any combination of accuracy, sensitivity, and specificity selected from those described above.

In some embodiments, the invention discloses a method for diagnosing autism with accuracy, sensitivity, and/or specificity as described in the example included herewith.

For any method disclosed herein that includes discrete steps, the steps may be conducted in any feasible order. And, as appropriate, any combination of two or more steps may be conducted simultaneously.

The above description is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Evidence of Metabolomic Phenotypes

With this example, blood plasma from 454 banked samples from two independent studies was used as a training and test set. Discovery-based metabolomic analysis was employed to reveal novel metabolic subtypes among individuals with ASD which can stratify subjects within the ASD spectrum. An algorithm was applied to identify metabolic features that describe distinct subpopulations of ASD subjects with a high positive predictive value using a cutoff threshold based on the abundance of those metabolic feature in Typically Developing (TD) individuals. The metabolite 3-Carboxy-4-methyl-5-propyl-2-furanpropionic (CMPF) was identified as a potential biomarker of a metabolic subtype in ASD. CMPF was elevated 6 times more than the median level in TD individuals in 12% of the subjects with ASD across the two studies with a specificity and positive predictive value near 100%. Elevated CMPF was not associated with increases in other uremic toxins and was more likely to occur in individuals taking omega-3 supplements. This study demonstrates an approach where broad metabolic profiles can be mined for potential metabolites of diagnostic value in the diagnosis and treatment of those with ASD.

This example reports the diagnostic value of a metabolite revealed by metabolic profiling across two studies. Metabolic profiles were generated from 454 banked plasma samples from subjects enrolled in a study performed at the University of California-Davis Medical Investigation of Neurodevelopmental Disorders (MIND) Institute and another at the Arkansas Children's Hospital Research Institute (ACHRI) to discover and validate novel metabolic subtypes. It was previously demonstrated that metabolomic analysis can identify predictive metabolic signatures of ASD (West et al., 2014, PLoS One; 9(11):e112445). This example further refines this approach. And, the results indicate that metabolomic analysis can reveal novel metabolic subtypes among individuals with ASD which can stratify subjects within the ASD spectrum and that are diagnostically disparate from TD.

The samples broadly profiled in this example included blood plasma from 258 subjects recruited by the MIND for the Autism Phenome Project (APP) using an untargeted metabolomic analysis to identify metabolites that could distinguish ASD subpopulations or metabolic subtypes with a high positive predictive value and specificity from TD children using simple discriminatory thresholds. The metabolites and discriminatory thresholds were identified from profiling the APP subjects and then were evaluated in 196 subjects recruited by ACHRI for the Integrated Metabolic and Genomic Endeavor (IMAGE) study to confirm the repeatability of the metabolic subtype. The discovered metabolites were utilized as diagnostic biomarkers for a specific metabolic subtype that can stratify individuals within the spectrum of autism. These ASD subtypes, based on an individual's biochemical profile, potentially offer the opportunity for more individualized treatment including modified diet, dietary supplements and pharmacological therapy. These findings will also begin to identify novel drug targets for the development of new therapies tailored to specific ASD subtypes.

Using this experimental approach, the metabolite CMPF was identified through MS-MS analysis, which describes a metabolic subtype. CMPF is a uremic toxin of unknown origin in humans that is likely derived from dietary furans (Spiteller, 2005, Lipids; 40(8):755-71). Individuals within the subtype exhibit elevated CMPF levels that are increased by at least six fold over mean abundance levels of the unaffected population (TD and ASD individuals not displaying elevated levels of CMPF). Increased CMPF identified in 14% of APP ASD subjects and 8% AHCRI of ASD subjects totaling 12% of the subjects with ASD across the two studies with a specificity and positive predictive value near 100%. Elevated CMPF levels were 3 times more prevalent within APP subjects taking omega-3 supplements, but CMPF levels were not directly attributed to supplementation itself. Elevated CMPF was not associated with increases in other uremic toxins.

### Methods

Clinical Study Subject Samples. Study information for the Autism Phenome Project (APP) and the Integrated Metabolic and Genomic Endeavor study (IMAGE) is summarized in Table 1.

APP Participants. Banked plasma samples were obtained from the Autism Phenome Project (APP). The study protocol was approved by the Institutional Review Board for the UC Davis School of Medicine, and parents of each subject provided written informed consent. Diagnostic instruments included the Autism Diagnostic Observation Schedule-Generic (ADOS-G) (Lord et al., 2000, J Autism Dev Disord; 30:205-223) and the Autism Diagnostic Interview-Revised (ADI-R) (Lord et al., 1994, J Autism Dev Disord; 24:659-685). All diagnostic assessments were conducted according to research standards.

Inclusion criteria for ASD were taken from the diagnostic definition of ASD in young children formulated and agreed upon by the Collaborative Programs of Excellence in Autism (Once et al., 2012, Biol Psychiatry; 72(12):1020-5).

Inclusion criteria for TD controls included developmental scores within two standard deviations of the mean on all subscales of the MSEL. Exclusion criteria for TD controls included a diagnosis of Mental Retardation, Pervasive Developmental Disorder or Specific Language Impairment, or any known developmental, neurological, or behavioral problems. TD children were screened and excluded for autism with the Social Communication Questionnaire (Berument et al., 1999, Br J Psychiatry; 175:444-451) (scores >11) (SCQ—Lifetime Edition). Participants were native English speakers, ambulatory, and had no suspected vision or hearing problems. The exclusion criteria for all subjects consisted of the presence of Fragile X or other serious neurological (for example, seizures), psychiatric (for example, bipolar disorder) or known medical conditions including autoimmune disease and inflammatory bowel diseases/celiac disease. Children with known endocrine, cardiovascular, pulmonary, liver, or kidney disease were excluded from enrollment in the study. Peripheral blood was obtained in a non-fasting state and collected in acid-citrate-dextrose Vacutainers (BD Biosciences, San Jose, Calif.). Blood samples were only obtained from children who had been free of any illness for at least 48 hours. The plasma was collected immediately by centrifugation for 10 min at 2100 rpm and stored in aliquots at 80° C.

IMAGE Participants. Banked blood samples from the IMAGE study (Integrated Metabolic and Genomic Endeavor) conducted at ACHRI were used a one cohort in this study. Children 3 to 10 years of age were diagnosed by trained pediatricians with a diagnosis of Autistic Disorder as defined by the Diagnostic and Statistical Manual of Mental Disorders, Fourth Edition (DSM-IV 299.0), the Autism Diagnostic Observation Schedule (ADOS, Lord et al., 1989, J Autism Dev Disord; 19(2): 185-212), and/or the Childhood Autism Rating Scales (CARS <30, Schopler et al., 1980, J Autism Dev Disord, 10(1): 91-103) were enrolled. Children previously diagnosed with other conditions on the autism spectrum (pervasive developmental disorder-not-otherwise-specified (PDD-NOS), childhood disintegrative disorder or rare genetic diseases associated with symptoms of autism such as fragile X, Rett syndrome, or tuberous sclerosis were not included in the study. Children with chronic seizure disorders, recent infection, and high dose vitamin or mineral supplements above the recommended daily allowance were excluded. Unaffected siblings and unrelated TD children ages 3 to 10 with no medical history of behavioral or neurologic abnormalities by parent report were the comparison groups in addition to unrelated TD children recruited locally. The protocol was approved by the Institutional Review Board at the University of Arkansas for Medical Sciences and all parents signed informed consent. Blood samples were collected from fasted individuals before 9:00 am into EDTA-Vacutainer tubes and immediately chilled on ice before centrifuging at 1,300×g for 10 min at 4° C. Aliquots of plasma were transferred into cryostat tubes and stored at −80° C.

TABLE 1
Summarized study information.

| Patient Samples | |
|---|---|
| APP | IMAGE |

| Diagnoses | TD | ASD | TD | ASD |
|---|---|---|---|---|
| Sample Size | 89 | 169 | 107 | 89 |
| Average age (y) | 3.1 | 3 | 6.1 | 5.4 |
| Sex (male, %) | 69 | 83 | 43 | 85 |

| | | |
|---|---|---|
| Dietary Status | Fed | Fasted |
| Blood Collection Tube | ACD | EDTA |
| Age Range | 2 to 4 years | 3 to 10 years |

Abbreviations:
TD, Typically Developing;
ASD Autism Spectrum Disorder;
ACD, Acid Citrate Dextrose;
EDTA Ethylenediaminetetraacetic acid.

Blood Plasma Sample Preparation. Samples were randomized into batches to maintain the proportion of age, gender, and diagnoses in each batch across batches prior to sample metabolite extractions. Subject plasma samples were prepared and extracted in methanol as described in West et al. (West et al., 2014, PLoS One; 9(11):e112445). Briefly, Small molecule metabolites were extracted from 50 μL plasma aliquots using 450 μL of 8:1 methanol:water solution at −20° C. The samples were evaporated to dryness and then solubilized in 45 μL of a 50:50 mixture of 0.1% formic acid in acetonitrile: 0.1% formic acid, also containing internal standards.

Mass Spectrometry. Metabolic profiling was performed using liquid chromatography (LC) coupled to high resolution mass spectrometry (HRMS) methods employing an Agilent 1290 liquid chromatography system coupled to an Agilent 6520 high resolution Quadrupole Time-of-Flight (QTOF) mass spectrometer. The samples were randomized prior to LC-HRMS analysis. Four separate methods were employed to analyze sample extracts, resulting in four separate data acquisitions per sample. Methods included both reverse phase (C8) and hydrophilic interaction (HILIC) chromatography with either negative or positive electrospray ionization (West et al., 2014, PLoS One; 9(11):e112445).

Metabolite chemical structure confirmation by LC-HRMS-MS. The chemical structures of CMPF, DHA, p-cresol sulfate, and indoxyl sulfate were confirmed using tandem mass spectrometry (LC-HRMS-MS) methods with chromatographic conditions identical to those used for their discovery. LC-HRMS-MS analyses were performed on an Agilent QTOF mass spectrometer for patient samples and/or, reference blood samples with collision energy conditions optimized to obtain the highest quality product ion spectra. The resulting product ion spectra were then compared to product ion spectra obtained for reference standards and/or spectra available in public spectral databases such as METLIN or MassBank.

### Data Analysis

LC-HRMS Data Preprocessing. Raw data from LC-HRMS analyses was converted to open source mzData files and processed using the R software library XCMS as described in West et al. (West et al., 2014, PLoS One; 9(11):e112445). The entire APP sample set was process together for the identification of APP mass features. The entire IMAGE sample set was processed together independent of the APP sample set for the identification of mass features. All abundance values were normalized to the spiked-in internal standard IBMX for comparison across APP and IMAGE clinical studies.

Subtype Analysis. Identification of metabolites that may describe a metabolic subtype was performed using the APP samples (training set) and validated in the independent IMAGE sample set (test set). A heuristic algorithm was used to identify metabolites that detect an affected subpopulation or metabolic subtype of ASD individuals exhibiting an extreme change in abundance with a high positive predictive value from the unaffected TD and ASD individuals using an abundance cutoff for a diagnostic threshold. For each mass feature, the diagnostic abundance thresholds to detect increased or decreased abundance were calculated using the three sigma or 99.7% rule based on the mean of TD plus or minus three times the standard deviation of TD individuals. The diagnostic threshold to detect increased or decreased abundance was applied to identify subjects that exceeded either threshold. A subject exceeding the diagnostic threshold value was scored as being ASD. The performance of the feature was ranked using a confusion matrix generated from the scored subjects (predicted diagnosis) compared to their true diagnosis. The performance metrics of positive predictive value and sensitivity (subtype prevalence) were used to select features in the APP sample set for evaluation in IMAGE sample set. The most predictive of the thresholds (detecting either increased or decreased abundance) in the APP study was carried forward for evaluation in the IMAGE test set. The diagnostic abundance threshold calculated from the normalized abundance value in the APP study was used to predict the subjects of the IMAGE study to validate the metabolic subtype.

Correlation Analysis. Spearman correlation analysis of mass features, partial correlation analysis and cohesive block analysis were performed using the R packages “igraph” (Csardi and Nepusz, 2006, “The igraph software package for complex network research,” InterJournal, Complex Systems 1695, available on the world wide web at igraph.org) and “pcit” (Nathan et al., 2010, Bioinformatics; 26(3) 411-413) to create groups of related mass features across LC-HRMS column chemistries and polarities.

Clinical Covariates. Independence of categorical APP clinical covariates with the subjects of the CMPF subtype was tested using the Chi-squared test statistic using an alpha of 0.05 to reject the null hypothesis. Independence of continuous APP clinical covariates with the subjects of CMPF subtype was tested using Welch T-tests. The correlation of continuous APP covariates was evaluated using Pearson correlations. Statistical analysis was performed using the statistical programming language R.

### Results

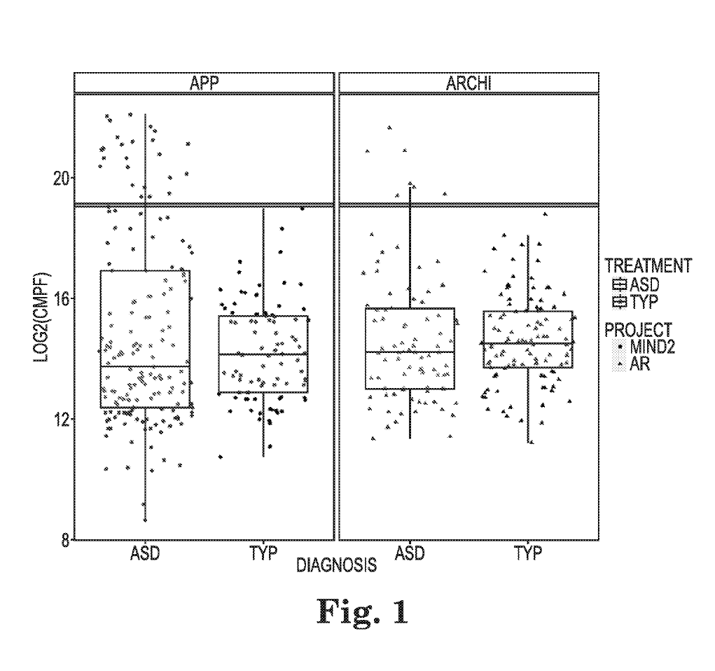

Identification of the metabolite CMPF associated identified a metabolic subtype in autism. A heuristic approach was applied to identify metabolic features able to discriminate subpopulations of individuals with ASD that exhibit profound metabolic perturbations within the APP study. This approach, by design, identifies metabolites which can produce a diagnostic threshold able to identify ASD individuals with extreme abundance values of the metabolite creating a metabolic subtype (affected population) and an unaffected population of ASD and TD individuals with “normal” abundance levels. Features that identified ASD subjects within the subtype population with a PPV >0.90 and had a subtype prevalence (sensitivity) among ASD subjects greater than 5% where considered as potential biomarkers for further evaluation. A total of 39 (1.5%) mass features that met PPV and subtype prevalence requirements were identified from 2657 high quality mass features that had verified chromatographic signals. These features were further evaluated by Spearman correlation, partial correlation, and cohesive block analysis to create feature groups that may be related through ionization or metabolism across the four LC-HRMS methods. A group of 8 of these 39 features were found to be related and could be represented using a single mass feature that had substantial abundance (FIG. 1). The MS-MS fragmentation of the most abundant representative mass feature suggested a putative annotation of CMPF. The retention time and fragmentation pattern of the mass feature was compared to the retention time and fragmentation pattern of a purchased CMPF chemical standard. The mass feature RT and fragmentation pattern were consistent with chemicals standard indicating the mass feature was CMPF.

Diagnostic Performance of CMPF in the APP study. A threshold of CMPF abundance which identified subjects with increased CMPF levels above on the mean of TD subjects plus 3 standard deviations correctly identified 14% (24/179) of the ASD subjects in the APP clinical study without false positives (FIG. 1). The confusion matrix based performance metrics demonstrated that CMPF described a metabolic subtype with a prevalence (sensitivity) of 14% a specificity of 100%, and a positive predictive value (PPV) of 100% (Table 2). In the APP study, the change in abundance between the ASD and TD subjects was similar, but significant (Welch T-test p value=0.043, ASD mean=14.8, TYP mean=14.2). When the ASD population is stratified by high CMPF levels, highly significant differences emerge (Welch T-test p value of <2e-16), resulting in an affected-CMPF mean of 20.7 compared to the unaffected-CMPF mean of 14.2 on the log 2 scale. This represents a greater than 6-fold increase in CMPF in the CMPF-elevated subpopulation (6.8 fold when compared to all non-elevated subjects). Difference between ASD and TD in subjects with non-elevated levels of CMPF is small (˜15%) and not statistically significant. The observation that CMPF is marginally elevated in ASD when compared to TD, but greatly increased in a subpopulation of ASD that largely defines the difference in abundance between ASD and TD suggests that this approach has identified a CMPF threshold that describes a distinct metabolic subtype of ASD subjects with markedly increased CMPF.

TABLE 2
Confusion matrix from the heuristic training process applied to
the APP study.

APP
Truth

| Predicted | ASD | TYP |
|---|---|---|
| ASD | 24 | 0 |
| TYP | 145 | 89 |

Autism Prevalence in Study = 66%
Accuracy = 44%
Balanced Accuracy = 57%
Sensitivity = 14%
Specificity = 100%
Positive Predictive Value = 100%
Negative Predictive Value = 38%
Kappa = 0.1025

Confirmation of the CMPF subtype in the IMAGE study. The reproducibility of the CMPF subtype was evaluated in the independent IMAGE study subjects by applying the diagnostic threshold identified in the APP study to predict the diagnosis of the subjects of the IMAGE study. The IMAGE subjects were predicted with a sensitivity of 7.9%, specificity of 100%, PPV of 100% demonstrating the reproducibility of the subtype in an independent study (Table 3). CMPF did not exhibit a statistically significant difference (Welch p value=0.74) in means (1%) between ASD and TD subjects in the IMAGE subjects. Similar to the APP study, a significant difference (Welch T-test p value <4e-7) in CMPF levels was present when the subjects were stratified using the APP CMPF diagnostic threshold. The affected subpopulation with elevated CMPF (mean=20.3) exhibiting a 5.8 fold increase over the unaffected subpopulation with non-elevated CMPF (mean=14.4). The characteristics of the CMPF subtype are similar in the APP and IMAGE populations with respect to abundance levels, fold change and diagnostic performance (PPV, subtype prevalence) demonstrating the reproducibility of the metabolic subtype identified in the APP study in the independent IMAGE study.

TABLE 3
Confusion matrix of CMPF based predictions of the ARCHI
clinical study using the threshold determined in the APP study.

ARCHI
Truth

| Predicted | ASD | TYP |
|---|---|---|
| ASD | 7 | 0 |
| TYP | 82 | 107 |

Autism Prevalence in Study = 45%
Accuracy = 58%
Balanced Accuracy = 54%
Sensitivity = 8%
Specificity = 100%
Positive Predictive Value = 100%
Negative Predictive Value = 57%
Kappa = 0.085

Evaluation of the CMPF subtype using IMAGE based threshold. The robustness and reproducibility of the CMPF diagnostic threshold was evaluated by reversing the threshold training and independent test sets. The heuristic algorithm was applied to the IMAGE sample set to determine a diagnostic threshold for CMPF based on the distribution of TD and then applied to the APP set to predict the subjects. The thresholds are nearly identical between the two studies with the APP determine threshold being 19.12 and the IMAGE determined threshold being 19.04 (FIG. 1, top and bottom horizontal lines respectively). Applying the IMAGE determined threshold to predict the ASD subjects APP and IMAGE studies does not change the prediction outcomes for either study. The APP predictions result in a 100% PPV and 14% prevalence, as might be expected given the marked elevation of CMPF and similar threshold values in the two studies. Since the populations described by the IMAGE threshold are identical to the APP threshold metrics associated with CMPF are not different than those presented above for the APP and IMAGE studies. The similarity of the threshold between the two studies is likely due to equivalent CMPF mean abundance (APP 14.2, IMAGE 14.6, Welch T-Test pvalue 0.08) and standard deviations (APP 1.64, ARCHI 1.48) in the two the studies. These results indicated that the plasma levels of CMPF are very robust despite the many differences in the study designs.

Association of CMPF subtype with clinical covariates in the APP study. The association of APP study covariates were evaluated with respect to the CMPF metabolic subtype population and the unaffected ASD population. The following covariates had no association with CMPF levels: gender, age, head size, developmental quotient, severity of ASD, date/time of blood draw, medications, multivitamin supplementation, ADOS scores, Mullen scores, sleep disorders, or gastrointestinal symptoms (Table 4). A single statistically significant (Chi-squared p value 0.0035) association was identified with supplementation of omega-3 fatty acids. In the APP study, 35 subjects (34 ASD, 1 TD) were taking omega-3 supplements, 15 were among the 24 ASD individuals with elevated CMPF. Of the individuals reporting omega-3 supplementation, 20 did not exhibit elevated CMPF levels. Also, nine individuals that did not report taking omega-3 supplements exhibited elevated levels of CMPF. Individuals who were supplemented with omega-3 fatty acids such as fish oil, EPA, DHA, and flax seed oil were 3 times more likely to exhibit increased CMPF than expected.

TABLE 4
Tests of Independence of subject metadata covariates and the CMPF subtype.

| Hypothesis | p value | Test |
|---|---|---|
| ASD by subtype prediction for DQ | 0.628 | Welch T-Test |
| ASD by subtype prediction for TCV_height_ratio | 0.853 | Welch T-Test |
| ASD by subtype prediction for sa_total | 0.602 | Welch T-Test |
| ASD by subtype prediction for rbb_total | 0.886 | Welch T-Test |
| ASD by subtype prediction for ados_total | 0.725 | Welch T-Test |
| ASD by subtype prediction for ados_severity | 0.873 | Welch T-Test |
| ASD by subtype prediction for | 0.850 | Welch T-Test |
| MULLEN_scoresumm_fm_age_equiv | | |
| ASD by subtype prediction for | 0.824 | Welch T-Test |
| MULLEN_scoresumm_fm_t_score | | |
| ASD by subtype prediction for VDQ | 0.749 | Welch T-Test |
| ASD by subtype prediction for NVDQ | 0.506 | Welch T-Test |
| ASD by subtype prediction for CBCL_anxious_depressed_sum | 0.311 | Welch T-Test |
| ASD by subtype prediction for CBCL_anxious_depressed_t | 0.512 | Welch T-Test |
| ASD by subtype prediction for CBCL_sleep_problems_sum | 0.753 | Welch T-Test |
| ASD by subtype prediction for CBCL_sleep_problems_t | 0.582 | Welch T-Test |
| ASD by subtype prediction for CBCL_dsm_anxiety_sum | 0.616 | Welch T-Test |
| ASD by subtype prediction for CBCL_dsm_anxiety_t | 0.676 | Welch T-Test |
| ASD by subtype prediction for CBCL_dsm_adhd_sum | 0.352 | Welch T-Test |
| ASD by subtype prediction for CBCL_dsm_adhd_t | 0.422 | Welch T-Test |
| ASD by subtype prediction for CBCL_dsm_odd_sum | 0.746 | Welch T-Test |
| ASD by subtype prediction for CBCL_dsm_odd_t | 0.134 | Welch T-Test |
| ASD by subtype prediction for SensoryProfile_TOT_RS | 0.515 | Welch T-Test |
| ASD by subtype prediction for GI.sum | 0.892 | Welch T-Test |
| ASD by subtype prediction for Regression_Onset | 0.758 | Chi-Squared |
| ASD by subtype prediction for NEW_Mega_Subgroup | 0.709 | Chi-Squared |
| ASD by subtype prediction for abdominal_pain_current | 0.763 | Chi-Squared |
| ASD by subtype prediction for | 0.797 | Chi-Squared |
| gaseousness_or_bloating_sensat_c | | |
| ASD by subtype prediction for diarrhea | 0.841 | Chi-Squared |
| ASD by subtype prediction for constipation_current | 0.963 | Chi-Squared |
| ASD by subtype prediction for abdominal_pain_current_corr.1 | 0.826 | Chi-Squared |
| ASD by subtype prediction for | 0.825 | Chi-Squared |
| gaseousness_or_bloating_sensat_c_corr.1 | | |
| ASD by subtype prediction for diarrhea_corr.1 | 0.813 | Chi-Squared |
| ASD by subtype prediction for constipation_current_corr.1 | 0.957 | Chi-Squared |
| ASD by subtype prediction for gastrointestinal_dx | 1.000 | Chi-Squared |
| ASD by subtype prediction for seizure_like_activity | 0.928 | Chi-Squared |
| ASD by subtype prediction for recurrent_otitis_media | 1.000 | Chi-Squared |
| ASD by subtype prediction for reflux | 0.856 | Chi-Squared |
| ASD by subtype prediction for relfux_med_required | 1.000 | Chi-Squared |
| ASD by subtype prediction for GF | 0.045 | Chi-Squared |
| ASD by subtype prediction for CF | 0.122 | Chi-Squared |
| ASD by subtype prediction for GFCF | 0.072 | Chi-Squared |
| ASD by subtype prediction for Vitamins.and.Minerals | 0.103 | Chi-Squared |
| ASD by subtype prediction for omega.3.fish.flax.supplement. | 0.005 | Chi-Squared |
| ASD by subtype prediction for No.Illnesses.Reported | 0.953 | Chi-Squared |

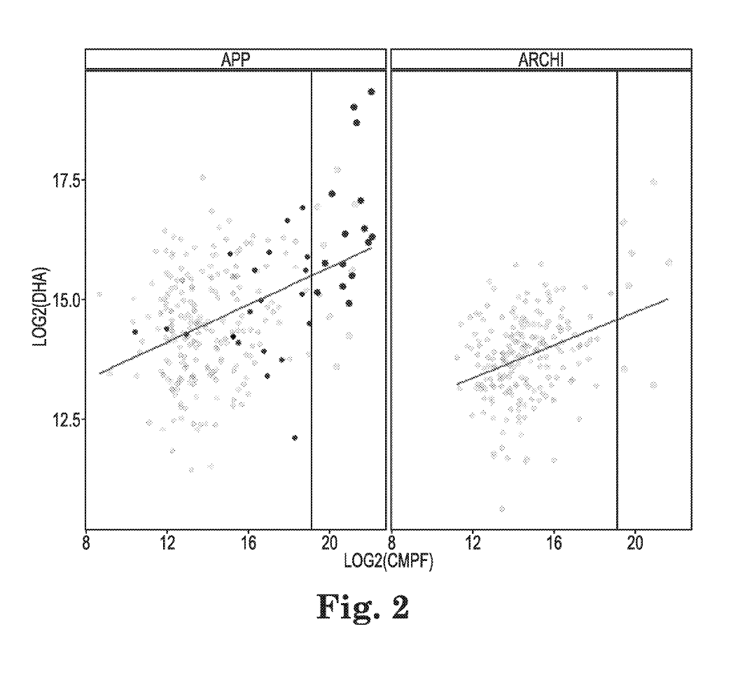

The relationship between omega-3 fatty acid DHA and CMPF was evaluated to understand whether there is a clear and direct relationship between omega-3 supplementation and CMPF levels. Information on omega-3 supplementation was available for the APP samples, but not available for IMAGE samples. To evaluate supplementation across studies, the omega-3 fatty acid DHA, a common ingredient in omega-3 supplements, was evaluated as a proxy for omega-3 fatty acid supplementation. DHA and CMPF abundance levels in both studies and CMPF levels were found to be moderately correlated (APP spearman correlation coefficient=0.31, p value=0.0002, IMAGE spearman correlation coefficient=0.26, p value=0.0002) with the levels of DHA in both studies (FIG. 2). The correlation of DHA with CMPF suggests that omega-3 supplements have an impact on CMPF levels, however, DHA levels do not separate the APP subjects with elevated CMPF from subjects with normal levels of DHA that occur in those not taking supplements. Further evaluation of CMPF levels in the APP subjects with respect to supplementation were completely confounded with the diagnosis of ASD as only a single TD individual reported supplementation. Supplementation in the APP study was associated with a 4.39 (21×) fold increase in CMPF levels (Welch T test p value <3e-10) and a 1.12 fold (2.2×) increase in the levels of DHA (Welch T test p value <4e-4). No differences in DHA levels were found with respect to diagnosis. DHA levels were increased in the elevated CMPF subtype populations by 1.80 fold in the APP (Welch T-test p value 0.03) and 1.65 fold in the IMAGE (Welch T-test p value <4e-6) studies. The increase in CMPF is much greater in the subtype population than the increase in DHA in those with elevated CMPF, 6 fold versus 1.5 fold, respectively. These results suggest that CMPF levels are impacted by omega-3 supplementation, but that CMPF levels are increased above what would be expected through supplementation alone in the elevated CMPF subtype population. Further evaluation of CMPF levels and omega-3 supplementation in TD populations are required to further understand the impact of omega-3 supplementation on CMPF levels and ASD diagnosis. One hypothesis is that individuals with high levels of CMPF are unable to properly metabolize and eliminate CMPF whether the source of CMPF is from the diet or from omega-3 supplementation or both thereby impacting abundance of CMPF as a function of improper metabolism of CMPF.

CMPF is the only uremic toxin that is elevated in ASD. Uremic toxins 3-indoxyl sulfate and p-cresol sulfate were evaluated in the APP and IMAGE subjects to determine if they are elevated in a manner similar to CMPF as would be expected if an undiagnosed kidney disease was present within the subtype. Differences in the measured levels of the 3-indoxyl sulfate and p-cresol sulfate did not identify a subpopulation of ASD subjects. Furthermore these uremic toxins were not correlated with CMPF across subjects or within the CMPF subtype. Uremic toxins were also tested for differences in means between ASD and TD and were found to have similar abundance levels (Table 5). The levels of 3-indoxyl sulfate and p-cresol sulfate were unchanged in the subpopulation of subjects with elevated CMPF (Table 5) showing that CMPF is altered in an independent manner from these uremic toxins. Combined, these results demonstrate that CMPF is elevated in a subpopulation of ASD while other uremic toxins show little change between ASD and TD or with respect to abnormal levels of CMPF.

TABLE 5
Changes in the abundance of CMPF and other uremic toxins with
respect to diagnosis and the CMP subtype.

| | | | Diagnosis | | CMPF |
|---|---|---|---|---|---|
| | | Diagnosis | ASD/TD | CMPF subtype | subtype |
| Metabolite | Study | log2(ASD/TD) | pvalue | log2(CMPF+/CMPF−) | pvalue |
| CMPF | APP | 0.61 | 0.043 | 6.78 | 2.8E−37 |
| CMPF | ARCHI | 0.092 | 0.33 | 5.82 | 3.7E−7 |
| p-Cresol Sulfate | APP | −0.27 | 0.18 | −0.03 | 0.99 |
| p-Cresol Sulfate | ARCHI | 0.21 | 0.26 | −0.09 | 0.9 |
| Indoxyl Sulfate | APP | −0.07 | 0.6 | −0.08 | 0.6 |
| Indoxyl Sulfate | ARCHI | 0.04 | 0.73 | −0.38 | 0.39 |

Abbreviations:
CMPF+, elevated CMPF;
CMPF− normal CMPF.

### Discussion

With this example, diagnostic biomarkers are mined from the metabolomic profiles of ASD and TD individuals that could describe metabolic subtypes within the spectrum of ASD. A heuristic analysis was applied to select mass features able to describe a subpopulation of individuals with ASD using simple diagnostic thresholds based on the values observed in TD individuals. Using this approach, we identified the metabolite CMPF as a potential biomarker able to discriminate a metabolic subtype comprising 14% of the individuals with ASD in the APP study samples with PPV=100%. These findings were confirmed using samples obtained from the independent IMAGE study using the diagnostic threshold identified in the APP study. Results from the APP and IMAGE sample analysis exhibited similar increases in CMPF over those with normal levels and subtype prevalence of 14% and 7% respectively with combined prevalence of 12%. The subtype was detected when the training and test sample sets were reversed with identical metrics indicating the robustness of the threshold. The fact the subtype was reproducible across studies in spite of differences in the study designs such as fed status, blood collection tube types, and age, indicates potential use of CMPF as a biomarker is robust and amenable to measurement in a range of settings.

Metabolism related to CMPF is poorly understood. Whether it is solely derived from the diet or is a product of human metabolism of dietary furans is unknown. There is evidence that CMPF is created in humans through the metabolism of dietary furans (Spiteller, 2005, Lipids; 40(8):755-71). CMPF is generally elevated in the diets of individuals who prefer fish, shell fish, and leafy green plants (Guertin et al., 2014, Am J Clin Nutr; 100(1):208-17; and Pallister et al., 2015, Twin Res Hum Genet; 18(6):793-805). This example found that elevated CMPF was associated with omega-3 fatty acid supplementation in the APP clinical study and that ASD subjects were three times more likely than expected to be taking supplements. It also found that CMPF levels were moderately associated with the common omega-3 fatty acid supplement DHA in both studies. However, many of the subjects with elevated CMPF had normal levels of DHA and many supplemented individuals had normal levels of CMPF. The relationship between fish oil based omega-3 supplementation and elevated CMPF has been previously established and leads to a 300% increase in plasma CMPF levels in two independent studies (Whal 1992 and Lankinen et al., 2015, PLoS One; 10(4):e0124379). A similar increase in CMPF was observed as the upper limit of CMPF in those with high fish diets (Guertin et al., 2014, Am J Clin Nutr; 100(1):208-17). CMPF levels in our study were increased in the ASD subpopulation above what has been associated with omega-3 supplementation. CMPF levels were increased greater than 6-fold in individuals with evaluated CMPF that comprise the metabolic subtype. Increased CMPF within the metabolic subtype may indicate that differences in furan fatty acid catabolism, metabolism or elimination of CMPF is altered and can be identified by measuring CMPF in these children.

Since CMPF is a uremic toxin and increases in CMPF are associated with both chronic kidney disease and uremias, the levels of uremic toxins was measured. CMPF is normally filtered out of the body by the kidneys and excreted in urine, but may also be eliminated by CYPs and through ROS related mechanisms (REF). It was found that changes observed in the uremic toxins 3-indoxyl sulfate and p-cresol sulfate were not correlated to those of CMPF. Based on these findings, it is likely that the elevated CMPF levels observed do not result from kidney disease, but altered kidney transport cannot be ruled out. The results of this example support the possibility that high levels of CMPF may be present due to an as yet unknown regulatory or biochemical mechanism. Accumulation of CMPF could arise from decreased alternative catabolic clearance pathways or altered detoxification pathways. For example, polymorphisms in two genes, one coding for an O-methyltransferease and one for a cytochrome P450 (CYP1A2) have been implicated in a slower rate of melatonin metabolism in ASD (Veatch et al., 2015, J Autism Dev Disord; 45(1):100-10). Both of these enzymes are associated with furan (such as CMPF) detoxification and could be further investigated for an association with the ASD CMPF subtype discovered here. In addition, it is known that gestational diabetes results in greatly elevated plasma CMPF and the mechanisms responsible for this accumulation are unknown (Prentice et al., Cell Metabolism; 19:653-666). There could be a common mechanism leading to the elevation of CMPF that becomes perturbed in a subpopulation of individuals with ASD leading to the increase of CMPF in a manner independent of Glutathione Detoxification Metabolism (GDM).

Elevated levels of CMPF are also observed in mothers with gestational diabetes. In rodent models, increased CMPF is associated with results from decreased biosynthesis of insulin as well as conversion of thyroid hormone thyroxine (T4) to triiodothyonine (T3) (Metabolism; 42(11): 1468-74). Interestingly gestational diabetes is associated with a 20% increase in the incidence of having a child born with ASD, which could indicate a link between CMPF levels and ASD in the maternal environment (Xiang et al., 2015, JAMA; 313(14): 1425-34). Several studies have found connections between decreased maternal thyroid hormone levels and ASD and ADHD disorders in children (Roman, 2007, J Neurol Sci; 262(1-2): 15-26; Roman et al., 2013, Ann Neurol; 74(5):733-42; and Yau et al., 2015, J Autism Dev Disord; 45(3):719-30). CMPF has not been studied previously in the blood of children with ASD, however Ming et al. determined that CMPF was elevated by 2 fold in a non-statistically significant manner in the urine of ASD compared to TD individuals, this change was found independent of GI problems (Ming et al., 2012, J Proteome Res; 11(12):5856-62). It is well established that CMPF can inhibit the organic anion transporters OAT3 and OAT1 that mediate brain to blood transport as well as transport in other tissues and organs (Tahara 2005). It is possible that increased CMPF could impact brain development both prenatally and postnatally through inhibition of OAT transport at the blood brain barrier. The receptor OAT1 is present in placenta mediates transport across it (Nigam et al., 2013, Physiol Rev; 95(1):83-123). The OAT3 transporter is expressed within the brain during a very small window of early development (Nigam et al., 2013, Physiol Rev; 95(1):83-123). The inhibition of these transporters is believed to be associated with neurological symptoms, including cognitive impairment and epilepsy, in individuals with renal failure (Costigan et al., 1996, Nephron; 73(2):169-73). The etiology of neurological symptoms associated with elevated CMPF can be hypothesized to be caused by build-up of toxic metabolic byproducts in the brain due to inhibition of OAT3 preventing normal transport of these byproducts out of the brain. The OAT3 transporter transports metabolites of dopamine, serotonin and norepinephrine as well as melatonin out of the brain (Ohtsuki et al, 2002, J Neurochem; 83(1):57-66). The accumulation of these metabolites could lead to neurotoxic affects as well as disruption of normal neural development.

Previously, discovery-based multivariate machine learning methods were applied to identify predictive molecular signatures (West et al., 2014, PLoS One; 9(11):e112445). This example demonstrates that a predictive metabolic signature is present within ASD that describes 12% of the ASD population with perfect specificity (PPV=100%). The novel approach to identify potentially diagnostic molecules has the advantage that it identifies biomarkers that have high predictive value for a subset of the diagnostic class (ASD in this case). As this approach is applied to identify additional diagnostic molecules, it gives rise to the opportunity to reveal additional metabolite subtypes and increase the overall sensitivity.

Expanding the paradigm described here to add additional subtypes requires a larger population of ASD patients to identify metabolic subtypes that are less prevalent. Identifying metabolic subtypes may have an additive effect in the proportion of individuals with ASD that can be identified through changes in metabolism. Additional metabolite subtypes may further describe the metabolism related to previously identified comorbidities in ASD potentially yielding insights into the mechanisms of ASD. Alternatively, additional subtypes may describe different metabolic paths to ASD. In this case, a combination of these subtypes may result in a combined diagnostic for a significant percentage of ASD individuals, leading to earlier diagnosis and potentially targeted interventions.

### Example 2

### Subtype Analysis: A New Approach

Metabolomics has the potential to identify predictive and actionable biomarker profiles from a child's inherited biochemistry as well as capture the interactions of the gut microbiome with dietary and environmental factors that contribute to ASD. Identification of common metabolic profiles in children with ASD creates an opportunity to develop metabolic based diagnostics that enable early diagnosis and identification of metabolic subtypes that can facilitate intervention and lead to a better understanding of the biochemical changes associated with ASD. Metabolic subtypes of ASD can be identified using discovery metabolomics that can stratify a subpopulation of individuals with ASD from the larger population of ASD and non-ASD individuals.

For example, as discussed in Example 1, 3-carboxy-4-methyl-5-propyl-2-furanpropionicacid (CMPF) is elevated in subset of ASD patients measured by multiple methods and represents a biomarker associated with a metabolic subtype of ASD.

This example identifies additional predictive metabolic signatures which distinguish ASD from typically developing (TD) children enrolled in the Autism Phenome Project (APP). This example has also identified metabolic subtypes of ASD as defined by differentially abundant metabolic features that can identify a subset of ASD individuals with a high positive predictive value and specificity.

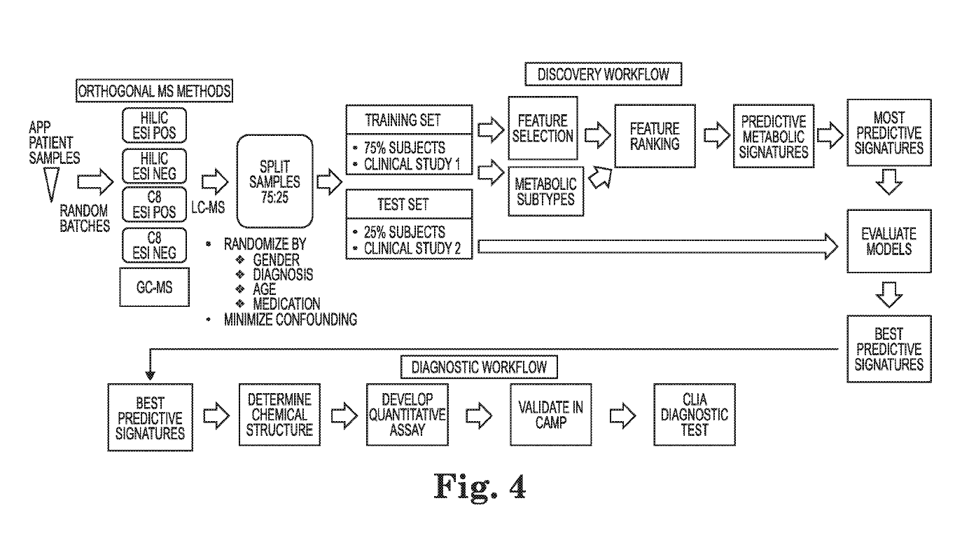

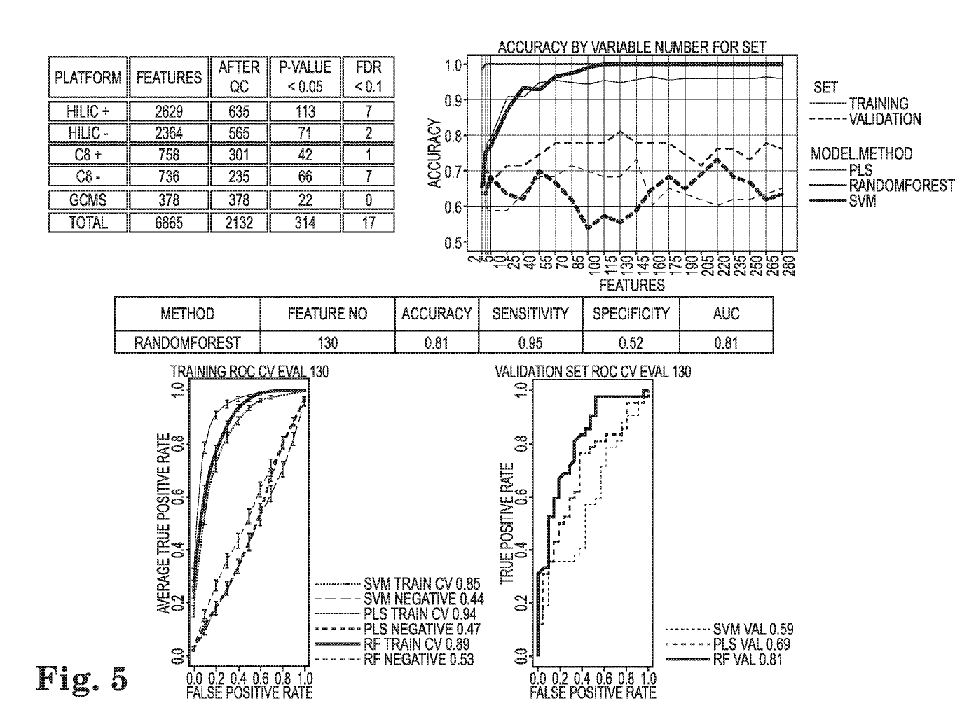

Methods: Plasma was obtained from 180 children with ASD at the time 1 assessment time point and from 93 age-matched TD children. Samples were analyzed using 4 orthogonal HILIC and C8 LC/HRMS-based methods as well as GC/MS. Data from the patient samples were split into a training set, utilized for identification of biomarkers, and an independent validation test set used for evaluation of the diagnostic signatures. Univariate, multivariate, machine learning and heuristic methods were applied to the training set to identify predictive metabolic features. The predictive molecular signatures were evaluated in the validation test set to determine their classification performance. This is shown in more detail in FIG. 4 and FIG. 5.

Results. Computational models were created that classified the ASD and TD samples in the validation set with a maximum accuracy of 79% and AUC of 0.80. Differentially abundant features (p value <0.05) from the models were identified as metabolites derived from multiple biochemical processes which included lysophospholipids, hormone sulfates, and amino acids. Two metabolites, 3-Carboxy-4-methyl-5-propyl-2-furanpropionic (CMPF) and an unknown metabolite related to CMPF exhibited a large differential abundance (>6 fold, p val <1e-6) in a subset of subjects with ASD. These metabolites discriminates 14% of the ASD population in the APP study and may describe a metabolic subtype of ASD.

The simple abundance threshold method for identifying one or more metabolites that identify subpopulations within a population of individuals with autism spectrum disorder (ASD), as described in the FIG. 4 and FIG. 5 can distinguish a subpopulation of 14% of the individuals with ASD and plasma based profiling of ASD vs TD children (2-4 years old) can be used to derive classification models with 79% accuracy.

Following the simple abundance threshold method, multiple metabolic subtypes have been identified in APP samples. These are listed in Table 6. The six subsets shown in Table 6 combined diagnose 51.4% of ASD subjects in APP. S2 to S6 without CMPF diagnose 45.3% of ASD subjects in APP.

As shown in FIG. 3, these subtypes may be combined for a diagnostic panel. Work is currently under way to assign chemical structures to the features of S2-S6. Discovery of additional subtypes will require larger patient populations to identify less frequent subtypes.

TABLE 6
Feature-Defined Subtypes in APP

| Subtype | Prevalence |
|---|---|
| CMPF | 14% |
| S2 | 23% |
| S3 | 16% |
| S4 | 13% |
| S5 | 10% |
| S6 | 5% |

Non-Targeted metabolomic profiling of children with ASD revealed predictive metabolic signatures able to discriminate individuals with ASD from TD individuals and suggests the presence of metabolic subtypes. Applying this paradigm to identify metabolic signatures associated with ASD and elucidating their biochemical implications may be useful in directing therapy on a personalized basis. Work is currently under way to compare these metabolic phenotypes with behavioral and neuroimaging data acquired in the APP. These results form the basis for additional work with the goals of developing diagnostic tests to detect ASD in children to improve their outcomes through personalized treatment, gaining new knowledge of biochemical mechanisms involved in ASD and identifying biomolecular targets for new modes of therapy.

### Example 3

### Confirmed Metabolites

Following procedures described in Examples 2 and 3, the metabolites listed in Table 7 have been confirmed as associated with ASD.

TABLE 7

| Metabolite | Method | Fold | P-Value | FDR |
|---|---|---|---|---|
| 2-Hydroxy-2-methylbutyric acid | C8 ESIneg | 0.84 | 0.006 | 0.282 |
| 3-Methyl-2-oxovaleric acid | C8 ESIneg | 0.87 | 0.013 | 0.412 |
| Salicylic acid | C8 ESIneg | 0.77 | 0.002 | 0.174 |
| Gentisic acid | C8 ESIneg | 0.71 | 0.000 | 0.036 |
| CMPF related metabolite | C8 ESIneg | 8.43 | 0.046 | 0.637 |
| DHEA sulfate | C8 ESIneg | 2.63 | 0.001 | 0.127 |
| Pregnenolone sulfate | C8 ESIneg | 1.71 | 0.000 | 0.029 |
| LysoPE(22:6) | C8 ESIpos | 1.38 | 0.000 | 0.028 |
| Glycine | HILIC ESIpos | 1.34 | 0.000 | 0.069 |
| L-Alanine or Sarcosine | HILIC ESIpos | 1.17 | 0.005 | 0.255 |
| Proline betaine | HILIC ESIpos | 0.72 | 0.000 | 0.069 |

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of diagnosing autism, the method comprising:
measuring the level of 3-carboxy-4-methyl-5-propyl-2-furanpropanoic acid (CMPF) in a biosample obtained from the individual;
wherein a level of CMPF at a level that is at least about six times or greater than the median level in TD individuals indicates autism; and/or
wherein a level of CMPF of at least about 2 μM or greater indicates autism.

2. The method of 1, further comprising measuring the level of one or more metabolites selected from 3-hydroxy-3-methylbutyric acid, 3-methyl-2-oxovaleric acid, salicylic acid, gentisic acid, a CMPF-related metabolite, DHEA sulfate, pregnenolone sulfate, LysoPE(22:6), glycine, 1-alanine, sacrosine, and/or proline betaine.

3. The method of claim 2,
wherein 3-hydroxy-3-methylbutyric acid of a fold change range of ASD/TD of about 0 to about 0.84, 3-methyl-2-oxovaleric acid of a fold change range of ASD/TD of about 0 to about 0.87, salicylic acid of a fold change range of ASD/TD of about 0 to about 0.77, gentisic acid of a fold change range of ASD/TD of about 0 to about 0.71, and/or proline betaine of a fold change range of ASD/TD of about 0 to about 0.72;
wherein the CMPF-related metabolite of a fold change range of TD/ASD of about 0 to about 0.12, DHEA sulfate of a fold change range of TD/ASD of about 0 to about 0.38, pregnenolone sulfate of a fold change range of TD/ASD of about 0 to about 0.58, LysoPE(22:6) of a fold change range of TD/ASD of about 0 to about 0.78, glycine of a fold change range of TD/ASD of about 0 to about 0.75, 1-alanine of a fold change range of TD/ASD of about 0 to about 0.86, sacrosine of a fold change range of TD/ASD of about 0 to about 0.85;
wherein 3-hydroxy-3-methylbutyric acid of a fold change range of ASD/TD is less than 0.84, 3-methyl-2-oxovaleric acid of a fold change range of ASD/TD of less than 0.87, salicylic acid of a fold change range of ASD/TD of less than 0.77, gentisic acid of a fold change range of ASD/TD of less than 0.71, the CMPF-related metabolite of a fold change range of ASD/TD of greater than about 8.43, DHEA sulfate of a fold change range of ASD/TD of greater than about 2.63, pregnenolone sulfate of a fold change range of ASD/TD of greater than about 1.71, LysoPE(22:6) of a fold change range of ASD/TD of greater than about 1.38, glycine of a fold change range of ASD/TD of greater than about 1.34, 1-alanine of a fold change range of ASD/TD of greater than about 1.17, sacrosine of a fold change range of ASD/TD of greater than about 1.17, and/or proline betaine of a fold change range of ASD/TD of greater than about 0.72; and/or
a measurement of 3-hydroxy-3-methylbutyric acid is less than 0.84 times the TD average or 1/0.84 times greater than the TD average, the measurement of 3-methyl-2-oxovaleric acid is less than 0.87 times the TD average or 1/0.87 times greater than the TD average, salicylic acid is less than 0.77 the TD average or 1/0.77 greater than the TD average, gentisic acid is less than 0.71 times the TD average or 1/0.71 times greater than the TD average, the CMPF-related metabolite is less than 8.43 times the TD average or 1/8.43 times greater than the TD average, DHEA sulfate is less than 2.63 times the TD average or 1/2.63 times greater than the TD average, pregnenolone sulfate is less than 1.71 times the TD average or 1/1.71 times greater than the TD average, LysoPE(22:6) is less than 1.38 times the TD average or 1/1.38 times greater than the TD average, glycine is less than 1.34 times the TD average or 1/1.34 times greater than the TD average, 1-alanine is less than 1.17 times the TD average or 1/1.17 times greater than the TD average, sacrosine is less than 1.17 times the TD average or 1/1.17 times greater than the TD average, and/or proline betaine is less than 0.72 times the TD average or 1/0.72 times greater than the TD average is indicative of autism.

4. A method of diagnosing autism, the method comprising:
measuring the level of one or more metabolites selected from 3-hydroxy-3-methylbutyric acid, 3-methyl-2-oxovaleric acid, salicylic acid, gentisic acid, a CMPF-related metabolite, DHEA sulfate, pregnenolone sulfate, LysoPE(22:6), glycine, 1-alanine, sacrosine, and/or proline betaine in a biosample obtained from the individual;
wherein 3-hydroxy-3-methylbutyric acid of a fold change range of ASD/TD of about 0 to about 0.84, 3-methyl-2-oxovaleric acid of a fold change range of ASD/TD of about 0 to about 0.87, salicylic acid of a fold change range of ASD/TD of about 0 to about 0.77, gentisic acid of a fold change range of ASD/TD of about 0 to about 0.71, and/or proline betaine of a fold change range of ASD/TD of about 0 to about 0.72;
wherein the CMPF-related metabolite of a fold change range of TD/ASD about 0 to about 0.12, DHEA sulfate of a fold change range of TD/ASD of about 0 to about 0.38, pregnenolone sulfate of a fold change range of TD/ASD of about 0 to about 0.58, LysoPE(22:6) of a fold change range of TD/ASD of about 0 to about 0.78, glycine of a fold change range of TD/ASD of about 0 to about 0.75, 1-alanine of a fold change range of TD/ASD of about 0 to about 0.86, sacrosine of a fold change range of TD/ASD of about 0 to about 0.85;
wherein 3-hydroxy-3-methylbutyric acid of a fold change range of ASD/TD is less than 0.84, 3-methyl-2-oxovaleric acid of a fold change range of ASD/TD of less than 0.87, salicylic acid of a fold change range of ASD/TD of less than 0.77, gentisic acid of a fold change range of ASD/TD of less than 0.71, the CMPF-related metabolite of a fold change range of ASD/TD of greater than about 8.43, DHEA sulfate of a fold change range of ASD/TD of greater than about 2.63, pregnenolone sulfate of a fold change range of ASD/TD of greater than about 1.71, LysoPE(22:6) of a fold change range of ASD/TD of greater than about 1.38, glycine of a fold change range of ASD/TD of greater than about 1.34, 1-alanine of a fold change range of ASD/TD of greater than about 1.17, sacrosine of a fold change range of ASD/TD of greater than about 1.17, and/or proline betaine of a fold change range of ASD/TD of greater than about 0.72; and/or
a measurement of 3-hydroxy-3-methylbutyric acid is less than 0.84 times the TD average or 1/0.84 times greater than the TD average, the measurement of 3-methyl-2-oxovaleric acid is less than 0.87 times the TD average or 1/0.87 times greater than the TD average, salicylic acid is less than 0.77 the TD average or 1/0.77 greater than the TD average, gentisic acid is less than 0.71 times the TD average or 1/0.71 times greater than the TD average, the CMPF-related metabolite is less than 8.43 times the TD average or 1/8.43 times greater than the TD average, DHEA sulfate is less than 2.63 times the TD average or 1/2.63 times greater than the TD average, pregnenolone sulfate is less than 1.71 times the TD average or 1/1.71 times greater than the TD average, LysoPE(22:6) is less than 1.38 times the TD average or 1/1.38 times greater than the TD average, glycine is less than 1.34 times the TD average or 1/1.34 times greater than the TD average, 1-alanine is less than 1.17 times the TD average or 1/1.17 times greater than the TD average, sacrosine is less than 1.17 times the TD average or 1/1.17 times greater than the TD average, and/or proline betaine is less than 0.72 times the TD average or 1/0.72 times greater than the TD average is indicative of autism.

5. A method of placing an individual within an autism subpopulation, the method comprising:
measuring the level of 3-carboxy-4-methyl-5-propyl-2-furanpropanoic acid (CMPF) in a biosample obtained from the individual;
wherein a level of CMPF at a level that is at least about six times or greater than the median level in TD individuals and/or a level of CMPF of at least about 2 μM or greater places the individual in a CMPF autism subpopulation.

6. The method of any one of claims 1 to 5 further comprising providing individualized treatment to the one or more individuals identified as belonging to the autism subpopulation.

7. The method of claim 6, wherein the individualized treatment comprises modified diet, dietary supplements, probiotic therapy, and/or pharmacological therapy.

8. The method of claim 6, wherein the individualized treatment comprises administration of a CMPF inhibitor and/or angiotensin II AT1 receptor blocker.

9. A method of placing an individual already clinically diagnosed with autism spectrum disorder (ASD) in an autism subset, the method comprising:
obtaining a biosample from the individual already clinically diagnosed with ASD;
quantifying the concentration amount of 3-carboxy-4-methyl-5-propyl-2-furanpropanoic acid (CMPF) in the biosample;
wherein if the concentration of CMPF is about 2 μM or greater and/or at least about six times or greater than the median level in TD individuals, then placing the individual in a CMPF autism subpopulation.

10. A method of diagnosing and treating autism in an individual, the method comprising:
obtaining a biosample from the individual;
quantifying the concentration amount of 3-carboxy-4-methyl-5-propyl-2-furanpropanoic acid (CMPF) in the biosample;
wherein if the concentration of CMPF in the biosample is about 2 μM or greater and/or at least about six times or greater than the median level in TD individuals, then administering an appropriate autism treatment.

11. A method of treating autism, the method comprising:
quantifying the concentration amount of 3-carboxy-4-methyl-5-propyl-2-furanpropanoic acid (CMPF) in a biosample obtained from an individual;
wherein CMPF concentrations is quantified using C18 (reverse phase) LC coupled with a triple quadrupole (QqQ) MS using electrospray ionization in the positive ion mode with analyte detection in the multiple reaction monitoring (MRM) mode and comprising a stable label internal standard and CMPF concentrations are measured distributed over a linear range of 0.05 to 100 μM;
wherein if the concentration of CMPF is about 2 μM or greater and/or at least about six times or greater than the median level in TD individuals, then administering an appropriate CMPF-associated ASD subset treatment.

12. A method of claim 10 or 11, further comprising quantifying the one or more metabolite indicative of ASD and/or an ASD subset at one or more time points after the initiation of treatment.

13. The method of claim 1, wherein the level of the one or more metabolites indicative of ASD and/or an ASD subset returns to TD levels after initiation of treatment.

14. A method comprising:
obtaining a biosample from a human subject; and
measuring the metabolite comprises 3-carboxy-4-methyl-5-propyl-2-furanpropanoic acid (CMPF) in the biosample;
wherein CMPF concentrations are determined using C18 (reverse phase) LC coupled with a triple quadrupole (QqQ) MS using electrospray ionization in the positive ion mode with analyte detection in the multiple reaction monitoring (MRM) mode and comprising a stable label internal standard, wherein CMPF concentrations are measured distributed over a linear range of 0.05 to 100 μM.

15. A method comprising measuring by mass spectrometry the levels of a plurality of metabolites in a biosample obtained from a human subject, wherein the plurality of metabolites comprises 3-carboxy-4-methyl-5-propyl-2-furanpropanoic acid (CMPF) and at least one metabolite selected from further comprising measuring the level of one or more metabolites selected from 3-hydroxy-3-methylbutyric acid, 3-methyl-2-oxovaleric acid, salicylic acid, gentisic acid, a CMPF-related metabolite, DHEA sulfate, pregnenolone sulfate, LysoPE(22:6), glycine, 1-alanine, sacrosine, proline betaine, 3-indoxylsulfate, p-cresol sulfate, and/or a 3-omega fatty acid metabolite.

16. A method of identifying a subpopulation within a population of individuals with autism spectrum disorder (ASD), the method comprising:
measuring the level of 3-carboxy-4-methyl-5-propyl-2-furanpropanoic acid (CMPF) in biosamples obtained from a population of ASD individuals; and
measuring the level of CMPF in biosamples obtained from a population of typically developing (TD) individuals;
comparing the level of CMPF in biosamples obtained from ASD individuals to the level of CMPF in biosamples obtained from TD individuals;
wherein a level of CMPF at a level that is at least about six times or greater than the median level in TD individuals places the ASD individuals in an autism subpopulation; and/or
wherein a level of CMPF at least about 2 μM or greater places the ASD individuals in an autism subpopulation.

17. The method of any one of claims 4 to 16, further comprising measuring the level of one or more metabolites selected from 3-hydroxy-3-methylbutyric acid, 3-methyl-2-oxovaleric acid, salicylic acid, gentisic acid, a CMPF-related metabolite, DHEA sulfate, pregnenolone sulfate, LysoPE(22:6), glycine, 1-alanine, sacrosine, and/or proline betaine.

18. The method of any one of claims 1 to 17, wherein measuring levels of CMPF in the biosample comprises mass spectrometry.

19. The method of claim 18, wherein mass spectrometry comprises gas chromatography mass spectrometry (GC-MS), and liquid chromatography mass spectrometry (e.g. LC-MS, LC-MS-MS, LC-MRM, LC-SIM, LC-SRM) using reverse phase liquid chromatography coupled to electrospray ionization in positive ion polarity (C8pos), reverse phase liquid chromatography coupled to electrospray ionization in negative ion polarity (C8neg), hydrophilic interaction liquid chromatography coupled to electrospray ionization in positive ion polarity (HILICpos), and/or hydrophilic interaction liquid chromatography coupled to electrospray ionization in negative ion polarity (HILICneg).

20. The method of claim 18, wherein mass spectrometry comprises C18 (reverse phase) LC coupled with a triple quadrupole (QqQ) MS using electrospray ionization in the positive ion mode with analyte detection in the multiple reaction monitoring (MRM) mode and comprising a stable label internal standard, wherein CMPF concentrations are measured distributed over a linear range of 0.05 to 100 μM.

21. The method of any one of claims 1 to 20, further comprising measuring the level of one or more additional non-CMPF uremic toxins; wherein the level of the one or more additional non-CMPF uremic toxins in the biosample obtained from the individuals in the autism subpopulation is similar to that in biosamples obtained from TD individuals.

22. The method of claim 21, wherein the one or more additional non-CMPF uremic toxin comprises 3-indoxyl sulfate and/or p-cresol sulfate.

23. The method of any one of claims 1 to 22, further comprising measuring the level of one or more 3-omega fatty acid metabolites.

24. The method of any one of claims 1 to 23, wherein the individual does not suffer from uremia, type 2 diabetes, and/or gestational diabetes.

25. The method of any one of claims 1 to 24, further comprising measuring the level of one or more metabolites selected from 3-hydroxy-3-methylbutyric acid, 3-methyl-2-oxovaleric acid, salicylic acid, gentisic acid, a CMPF-related metabolite, DHEA sulfate, pregnenolone sulfate, LysoPE(22:6), glycine, 1-alanine, sacrosine, and/or proline betaine.

26. The method of claim 25, wherein:
3-hydroxy-3-methylbutyric acid of a fold change range of ASD/TD of about 0 to about 0.84, 3-methyl-2-oxovaleric acid of a fold change range of ASD/TD of about 0 to about 0.87, salicylic acid of a fold change range of ASD/TD of about 0 to about 0.77, gentisic acid of a fold change range of ASD/TD of about 0 to about 0.71, and/or proline betaine of a fold change range of ASD/TD of about 0 to about 0.72;
the CMPF-related metabolite of a fold change range of TD/ASD of about 0 to about 0.12, DHEA sulfate of a fold change range of TD/ASD of about 0 to about 0.38, pregnenolone sulfate of a fold change range of TD/ASD of about 0 to about 0.58, LysoPE(22:6) of a fold change range of TD/ASD of about 0 to about 0.78, glycine of a fold change range of TD/ASD of about 0 to about 0.75, 1-alanine of a fold change range of TD/ASD of about 0 to about 0.86, sacrosine of a fold change range of TD/ASD of about 0 to about 0.85;
3-hydroxy-3-methylbutyric acid of a fold change range of ASD/TD is less than 0.84, 3-methyl-2-oxovaleric acid of a fold change range of ASD/TD of less than 0.87, salicylic acid of a fold change range of ASD/TD of less than 0.77, gentisic acid of a ASD/TD of less than 0.71, the CMPF-related metabolite of a fold change range of ASD/TD of greater than about 8.43, DHEA sulfate of a fold change range of ASD/TD of greater than about 2.63, pregnenolone sulfate of a fold change range of ASD/TD of greater than about 1.71, LysoPE(22:6) of a fold change range of ASD/TD of greater than about 1.38, glycine of a fold change range of ASD/TD of greater than about 1.34, 1-alanine of a fold change range of ASD/TD of greater than about 1.17, sacrosine of a fold change range of ASD/TD of greater than about 1.17, and/or proline betaine of a fold change range of ASD/TD of greater than about 0.72; and/or
a measurement of 3-hydroxy-3-methylbutyric acid is less than 0.84 times the TD average or 1/0.84 times greater than the TD average, the measurement of 3-methyl-2-oxovaleric acid is less than 0.87 times the TD average or 1/0.87 times greater than the TD average, salicylic acid is less than 0.77 the TD average or 1/0.77 greater than the TD average, gentisic acid is less than 0.71 times the TD average or 1/0.71 times greater than the TD average, the CMPF-related metabolite is less than 8.43 times the TD average or 1/8.43 times greater than the TD average, DHEA sulfate is less than 2.63 times the TD average or 1/2.63 times greater than the TD average, pregnenolone sulfate is less than 1.71 times the TD average or 1/1.71 times greater than the TD average, LysoPE(22:6) is less than 1.38 times the TD average or 1/1.38 times greater than the TD average, glycine is less than 1.34 times the TD average or 1/1.34 times greater than the TD average, 1-alanine is less than 1.17 times the TD average or 1/1.17 times greater than the TD average, sacrosine is less than 1.17 times the TD average or 1/1.17 times greater than the TD average, and/or proline betaine is less than 0.72 times the TD average or 1/0.72 times greater than the TD average places the individual in the autism subpopulation.

27. The method of any one of claims 1 to 26, wherein the individual has been previously diagnosed with autism spectrum disorder (ASD) and/or is undergoing treatment for autism.

28. The method of any one of claims 1 to 26, wherein the individual has not been previously diagnosed with autism spectrum disorder (ASD).

29. The methods of any one of claims 1 to 28, wherein the biosample comprises cerebrospinal fluid, brain tissue, amniotic fluid, blood, serum, plasma, amniotic fluid, urine, breath condensate, sweat, saliva, tears, hair, cell membranes, and/or vitreous humour.

30. The method of any one of claims 1 to 29, wherein the biosample comprises plasma.

31. The method of any one of claims 1 to 30, wherein the subject is an adult, is a teenager, is less than 13 years of age, is less than 10 years of age, is less is about 6 years of age, less than about 5 years of age, less than about 4 years of age, less than about 3 years of age, less than about 2 years of age, less than about 18 months of age, less than about 1 year of age, about 1 to about 6 years of age, about 1 to about 5 years of age, about 1 to about 4 years of age, about 1 to about 2 years of age, about 2 to about 6 years of age, about 2 to about 4 years of age, or about 4 to about 6 years of age.

32. A metabolomic signature for a subset of autism, the metabolomic signature comprising 3-carboxy-4-methyl-5-propyl-2-furanpropanoic acid (CMPF) at a concentration of at least about six times or greater than the median level in TD individuals and/or at least about 2 μM or greater.

33. The metabolomic signature for autism of claim 32, the metabolomic signature further comprising at least one metabolite selected from 3-hydroxy-3-methylbutyric acid, 3-methyl-2-oxovaleric acid, salicylic acid, gentisic acid, a CMPF-related metabolite, DHEA sulfate, pregnenolone sulfate, LysoPE(22:6), glycine, 1-alanine, sacrosine, and/or proline betaine.

34. The metabolomic signature for autism of claim 33, the metabolomic signature comprising:
3-hydroxy-3-methylbutyric acid of a fold change range of ASD/TD of about 0 to about 0.84, 3-methyl-2-oxovaleric acid of a fold change range of ASD/TD of about 0 to about 0.87, salicylic acid of a fold change range of ASD/TD of about 0 to about 0.77, gentisic acid of a fold change range of ASD/TD of about 0 to about 0.71, and/or proline betaine of a fold change range of ASD/TD of about 0 to about 0.72;
the CMPF-related metabolite of a fold change range of TD/ASD of about 0 to about 0.12, DHEA sulfate of a fold change range of TD/ASD of about 0 to about 0.38, pregnenolone sulfate of a fold change range of TD/ASD of about 0 to about 0.58, LysoPE(22:6) of a fold change range of TD/ASD of about 0 to about 0.78, glycine of a fold change range of TD/ASD of about 0 to about 0.75, 1-alanine of a fold change range of TD/ASD of about 0 to about 0.86, sacrosine of a fold change range of TD/ASD of about 0 to about 0.85;
3-hydroxy-3-methylbutyric acid of a fold change range of ASD/TD is less than 0.84, 3-methyl-2-oxovaleric acid of a fold change range of ASD/TD of less than 0.87, salicylic acid of a fold change range of ASD/TD of less than 0.77, gentisic acid of a fold change range of ASD/TD of less than 0.71, the CMPF-related metabolite of a fold change range of ASD/TD of greater than about 8.43, DHEA sulfate of a fold change range of ASD/TD of greater than about 2.63, pregnenolone sulfate of a fold change range of ASD/TD of greater than about 1.71, LysoPE(22:6) of a fold change range of ASD/TD of greater than about 1.38, glycine of a fold change range of ASD/TD of greater than about 1.34, 1-alanine of a fold change range of ASD/TD of greater than about 1.17, sacrosine of a fold change range of ASD/TD of greater than about 1.17, and/or proline betaine of a fold change range of ASD/TD of greater than about 0.72; and/or
3-hydroxy-3-methylbutyric acid is less than 0.84 times the TD average or 1/0.84 times greater than the TD average, the measurement of 3-methyl-2-oxovaleric acid is less than 0.87 times the TD average or 1/0.87 times greater than the TD average, salicylic acid is less than 0.77 the TD average or 1/0.77 greater than the TD average, gentisic acid is less than 0.71 times the TD average or 1/0.71 times greater than the TD average, the CMPF-related metabolite is less than 8.43 times the TD average or 1/8.43 times greater than the TD average, DHEA sulfate is less than 2.63 times the TD average or 1/2.63 times greater than the TD average, pregnenolone sulfate is less than 1.71 times the TD average or 1/1.71 times greater than the TD average, LysoPE(22:6) is less than 1.38 times the TD average or 1/1.38 times greater than the TD average, glycine is less than 1.34 times the TD average or 1/1.34 times greater than the TD average, 1-alanine is less than 1.17 times the TD average or 1/1.17 times greater than the TD average, sacrosine is less than 1.17 times the TD average or 1/1.17 times greater than the TD average, and/or proline betaine is less than 0.72 times the TD average or 1/0.72 times greater than the TD average.

35. The metabolomic signature for autism of any one of claims 32 to 34, wherein CMPF concentrations are determined using C18 (reverse phase) LC coupled with a triple quadrupole (QqQ) MS using electrospray ionization in the positive ion mode with analyte detection in the multiple reaction monitoring (MRM) mode, and comprising a stable label internal standard wherein CMPF concentrations are measured distributed over a linear range of 0.05 to 100 μM.

36. A simple abundance threshold method of identifying one or more metabolites identifying a subpopulation within a population of individuals with autism spectrum disorder (ASD), or a method of identifying a subpopulation within a population of individuals with ASD, the method comprising:
measuring the levels of one or more features (for example, metabolites, putative-metabolites, unknown metabolites, proteins, and/or RNA) in two populations;
determining in one population an optimal upper threshold for each feature, wherein the upper threshold represents a level of the feature wherein all except 1% of subjects with a TD diagnosis has a level of feature which is below the threshold;
counting the number of subjects with a diagnosis of ASD which have feature levels above the upper threshold and saving as a hypothetical diagnostic features where this count is above about 6% of the total number of ASD subjects;
repeating the above steps for all features wherein a lower threshold is used which represents a level for which all but one TD subject has levels above the lower threshold;
saving as a hypothetical diagnostic every feature for which about 6% or greater of ASD subjects have feature levels below the lower threshold;
creating a multitude of feature ratios for each hypothetical diagnostic by dividing the level determined for each subject by a set of normalizing features (this set can be all features or a selected set of features or determined metabolites or putative metabolites);
determining ratio optimal thresholds for each feature;
determining the percent of ASD subjects which have ratios above or below the thresholds, wherein ratios which distinguish the greatest number of ASD subjects are saved as diagnostic ratio;
using a second population of subjects wherein the age, demographics and collection conditions (for example fasted or non-fasted) can be the same or different from the first study; and
determining the performance of each diagnostic ratio using the same optimal threshold which was determined in the first population of subjects;
wherein ratio diagnostics which perform with greater than about 90% specificity and about 6% sensitivity (or any performance requirements one sees fit) reveal features which define a subtype in ASD.

37. The method of claim 36, wherein one or more features comprises a confirmed metabolite.

38. The method of claim 36 or 37, wherein one or more features comprises a putative metabolites as determined by matching a library of features associated with known metabolites on mass and retention time.

39. The method of any one of claims 36 to 38, wherein the first population and the second population comprise subsets of a single study.

40. The method of any one of claims 36 to 39, wherein the first population and the second population comprise two independent studies.

41. The method of any one of claims 36 to 40, wherein feature levels and/or and diagnostic ratios are determined by the same or different techniques.

42. The method of any one of claims 36 to 41, wherein feature levels and/or and diagnostic ratios are determined using mass spectrometry.

43. The method of claim 42, wherein mass spectrometry comprises gas chromatography mass spectrometry (GC-MS), and liquid chromatography mass spectrometry (e.g. LC-MS, LC-MS-MS, LC-MRM, LC-SIM, LC-SRM) using reverse phase liquid chromatography coupled to electrospray ionization in positive ion polarity (C8pos), reverse phase liquid chromatography coupled to electrospray ionization in negative ion polarity (C8neg), hydrophilic interaction liquid chromatography coupled to electrospray ionization in positive ion polarity (HILICpos), and/or hydrophilic interaction liquid chromatography coupled to electrospray ionization in negative ion polarity (HILICneg).

44. The method of claim 42, wherein mass spectrometry comprises C18 (reverse phase) LC coupled with a triple quadrupole (QqQ) MS using electrospray ionization in the positive ion mode with analyte detection in the multiple reaction monitoring (MRM) mode comprising a stable label internal standard wherein CMPF concentrations are measured distributed over a linear range of 0.05 to 100 μM.

45. The method of any one of claims 36 to 44 wherein selected features used in the denominator of the diagnostic ratio are selected to be non-complementary to the numerator or hypothetical diagnostic feature (i.e. is not itself a hypothetical diagnostic feature).

46. The method of any one of claims 36 to 45, wherein the selected features used in the denominator are determined to be hypothetical diagnostics which when used in the denominator improve the diagnostic performance of the ratio (i.e. complementary to the feature in the numerator).

47. The method of any one of claims 36 to 46, wherein one or more metabolite(s) used for the denominator is a spiked-in agent.

48. The method of any one of claims 36 to 47, wherein the optimal upper threshold is defined as the level of feature for which all ASD or DD subjects are below the threshold and the optimal lower threshold is defined as the level of feature for all ASD or DD subjects which is above the threshold.

49. The method of any one of claims 36 to 48, wherein the optimal lower and upper thresholds are:
based on measures of dispersion population (for example, variance, IQR, MAD, CV, standard deviation, standard error and/or other statistical means) of the mean or median of the non-ASD;
based on measures of dispersion population (for example, variance, IQR, MAD, CV, standard deviation, standard error and/or other statistical means) of the mean or median of the ASD population;
based on the upper and lower quantiles of the non-ASD population;
based on the upper and lower quantiles of the ASD population;
based on a measure of statistical distance of the subjects with ASD to non-ASD subjects;
based on a standard score or standardized variable of non-ASD subjects;
based on a standard score or standardized variable of ASD subjects; or
based on ROC AUC.

50. The method of any one of claims 36 to 49, wherein:
the minimum percentage sensitivity required for the determination of a hypothetical diagnostic comprises about 3%, about 4%, about 5%, about 7%, about 8%, about 9%, about 10%, about 11%, about 12%, about 13%, about 14%, about 15%, about 16%, about 17%, about 18%, about 19%, or about 20%, rather than about 6%;
wherein the ratio diagnostics perform with greater than at least about 95% specificity, at least about 96% specificity, at least about 97% specificity, at least about 98% specificity, or at least about 99% specificity; and/or
wherein the ratio diagnostics perform with at least about 75% specificity, at least about 80% specificity, at least about 85% specificity, at least about 86% specificity, at least about 87% specificity, at least about 88% specificity, or at least about 89% specificity, rather than greater than about 90% specificity.
